SEQUENCE LISTING (79 sequences)

SEQ ID NO: 1 - protein, 393 aa, Lycopersicon esculentum
MPGIKGPSDYSREPPRHPSLKINAKEPFNAEPTRSALISSYVTPVDFFYKRNHGPIPVVDDIERYSVSLSGLIKNSKDLFMKDICKLPKYTVTATLQCAGNRRTAMSKSRTVKGVGWDIAALGNAVWGGAKLADVLELVGIPYLASITQSGGKHVEFVSIDKCKEENGGPYKASIPLSQATNPEADVLLAYEMNGEPLNRDHGYPLRVVVPGVIGARSVKWLDSINIIAEECKGFFMQKDYKMFPPTVNWDNINWSTRRPQMDFPVQSAICSLEDVSVVKHGKINIKGYALSGGGRGIERVDVSIDGGKTWEEATRYQRTGVPYIADDSSSDRWAWVFFEAEANIPQSAEIVAKAVDISANVQPESIGSVWNLRGILNTSWHRVHVRVGQANL

SEQ ID NO: 2 - DNA, 5122 bp, Artificial sequence
  pRITA reporter plasmid
gcggccgcatgcatatgtcgacctgcagacgcgtctcgaggaattcggtaccccgggttcgaaatcgatcaagcttggatccgggcccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaatcctagcttgatattctatagtgtcacctaaatctgcggccgctgaccaagtcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggaaattgtaaacgttaatattttgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggatgccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttggccaagtcggcctctaatacgactcactatagggagctc

SEQ ID NO: 3 - DNA, 30 bp, Artificial sequence
  Single strand DNA oligonucleotide
cgggatccctcgaggctcgttcggtcaaat

SEQ ID NO: 4 - DNA, 32 bp, Artificial sequence
  Single strand DNA oligonucleotide
ccatcgatgaattcctttctatcccgcgtcca

SEQ ID NO: 5 - DNA, 25 bp, Artificial sequence
  Single strand DNA oligonucleotide
gggctttgacatctttgaagaaaac

SEQ ID NO: 6 - DNA, 28 bp, Artificial sequence
  Single strand DNA oligonucleotide
tcaattgggataatatcaactggtcctc

SEQ ID NO: 7 - DNA, 25 bp, Artificial sequence
  Single strand DNA oligonucleotide
aaaacttacattcttggcagcagtg

SEQ ID NO: 8 - DNA, 28 bp, Artificial sequence
  Single strand DNA oligonucleotide
tcaattgggataatatcaactggtcctc

SEQ ID NO: 9 - DNA, 34 bp, Artificial sequence
  Single strand DNA oligonucleotide
agactcgagtatgaccttgggatatggtcctgtc

SEQ ID NO: 10 - DNA, 35 bp, Artificial sequence
  Single strand DNA oligonucleotide
tccaagctttcttctttcgaggaggagataccgag

SEQ ID NO: 11 - DNA, 27 bp, Artificial sequence
  Single strand DNA oligonucleotide
aggaaacagctatgaccatgattacga

SEQ ID NO: 12 - DNA, 27 bp, Artificial sequence
  Single strand DNA oligonucleotide
tttgtggtagacggaggtatacgagtg

SEQ ID NO: 13 - DNA, 60 bp, Artificial sequence
  Single strand DNA oligonucleotide
gctgtcaacgatacgctacgtaacggcatgacagtgtttttttttttttttttttttttt

SEQ ID NO: 14 - DNA, 20 bp, Artificial sequence
  Single strand DNA oligonucleotide
caagtcacacagcaccgttt

SEQ ID NO: 15 - DNA, 25 bp, Artificial sequence
  Single strand DNA oligonucleotide
gctgtcaacgatacgctacgtaacg

SEQ ID NO: 16 - DNA, 1581 bp, Lycopersicon esculentum
caagtcacacagcaccgtttaatttccaattgcatgaagagtataacctttctccacagcctcaaattcgtcaggtagtagggccgctccaatatttgtacaaatcttcttcatctacctcgcactcacacctagcaggactactgtgaagccggcggcaaaggaaactgagaaaatgcctgggattaaagggccttccgattattcgcgagaaccccctcgccatccttctcttaaaattaatgccaaggaaccctttaatgctgagccaactcgttcagctctgatttcatcttatgtcactcctgttgatttcttttacaagagaaaccacggacccataccagtagtggatgacatagagaggtattccgtttctctgagtggtcttataaaaaattccaaagacttgtttatgaaggatatttgtaagcttccaaaatatacggttactgccactttacagtgtgctggtaacagaagaactgctatgagtaagagtcgaacagtgaaaggagttggttgggatattgctgctttaggaaatgctgtttggggtggagccaaattggcagatgttctagaattagttggaataccttatttggcaagtatcacacaatctggaggaaaacatgtggaatttgtgagcattgacaagtgtaaggaggaaaatggaggtccttataaggcatcaattccattgagtcaggctacaaaccctgaagctgatgttctgctcgcttatgagatgaatggagagccacttaatagggatcatggctatccactgcgtgtggttgtccctggtgtgataggtgcccgttcagttaagtggcttgattccatcaatatcattgccgaagaatgcaagggattctttatgcaaaaagactacaagatgtttccaccaacagtgaattgggataacatcaactggtctactaggagaccgcagatggactttcctgttcagagcgcaatatgttccctggaggatgtgagtgttgtaaagcatggaaagataaacatcaaggggtacgcattatcaggaggtgggcgtggcatagaaagggtagacgtgtctatagatggtggtaaaacttgggaggaagccaccagataccagagaactggtgttccatatattgctgatgattcaagcagtgacagatgggcatgggtcttttttgaagctgaggcaaatattccccagagtgcggaaatagttgctaaagcggtggatatatctgcaaatgtccaacctgaaagtataggttctgtctggaatctgagaggaatcctgaacacctcatggcatcgggttcatgtccgagttggtcaagcaaatctttaggctgtgtttggtgtgttgaagcggatgacagtaatcagcatatcaggccatggttgtttgtatccttcttagcttaaaatgaaaatagcatggtggtttgcgtcttcctagcttaaaatgaaaataaaatgttctcttgatgttgtctcgaaatacttacggaaataataataatttggaagtgattgcatcttgacaattaaaaaaaaaaaaaaaaaaaaaaa

SEQ ID NO: 17 - DNA, 30 bp, Artificial sequence
  Single strand DNA oligonucleotide
cgggatccctcgagagacttgtttatgaag

SEQ ID NO: 18 - DNA, 32 bp, Artificial sequence
  Single strand DNA oligonucleotide
ccatcgatgaattccttacacttgtcaatgct

SEQ ID NO: 19 - DNA, 25 bp, Artificial sequence
  Single strand DNA oligonucleotide
gggctttgacatctttgaagaaaac

SEQ ID NO: 20 - DNA, 27 bp, Artificial sequence
  Single strand DNA oligonucleotide
ggatattgctgctttaggaaatgctgt

SEQ ID NO: 21 - DNA, 25 bp, Artificial sequence
  Single strand DNA oligonucleotide
aaaacttacattcttggcagcagtg

SEQ ID NO: 22 - DNA, 27 bp, Artificial sequence
  Single strand DNA oligonucleotide
ggatattgctgctttaggaaatgctgt

SEQ ID NO: 23 - DNA, 29 bp, Artificial sequence
  Single strand DNA oligonucleotide
acactcgagatgcctgggattaaagggcc

SEQ ID NO: 24 - DNA, 30 bp, Artificial sequence
  Single strand DNA oligonucleotide
tacgaattcctaaagatttgcttgaccaac

SEQ ID NO: 25 - DNA, 27 bp, Artificial sequence
  Single strand DNA oligonucleotide
atcattgcgataaaggaaaggctatca

SEQ ID NO: 26 - DNA, 25 bp, Artificial sequence
  Single strand DNA oligonucleotide
gaataatcggaaggccctttaatcc

SEQ ID NO: 27 - protein, 16 aa, Artificial sequence
  Tomato and Arabidopsis SO synthetic polypeptide
RHPSLKINAKEPFNAE

SEQ ID NO: 28 - DNA, 27 bp, Artificial sequece
  Single strand DNA oligonucleotide
aaggatttcgtgtgaagaaggaagaac

SEQ ID NO: 29 - DNA, 25 bp, Artificial sequece
  Single strand DNA oligonucleotide
caactcatttttcgcacgcttatct

SEQ ID NO: 30 - DNA, 27 bp, Artificial sequece
  Single strand DNA oligonucleotide
tctcaagagattgggagcaaatatgaa

SEQ ID NO: 31 - DNA, 28 bp, Artificial sequece
  Single strand DNA oligonucleotide
gaactgttccagtacatttttcgctttt

SEQ ID NO: 32 - DNA, 26 bp, Artificial sequece
  Single strand DNA oligonucleotide
gtgttcaaaaggctgaggattggtta

SEQ ID NO: 33 - DNA, 27 bp, Artificial sequece
  Single strand DNA oligonucleotide
ctcttggaatccttgttcctgattctc

SEQ ID NO: 34 - DNA, 27 bp, Artificial sequece
  Single strand DNA oligonucleotide
ttacatcctcaagagtgccacaaggac

SEQ ID NO: 35 - DNA, 28 bp, Artificial sequece
  Single strand DNA oligonucleotide
cgaaggttagcccaatgtctagttgata

SEQ ID NO: 36 - DNA, 26 bp, Artificial sequece
  Single strand DNA oligonucleotide
agactcaccaacagcttgaccaattt

SEQ ID NO: 37 - DNA, 26 bp, Artificial sequece
  Single strand DNA oligonucleotide
cacctagtccatcatccgagctagag

SEQ ID NO: 38 - DNA, 25 bp, Artificial sequece
  Single strand DNA oligonucleotide
aagagtggggatttttccagcttgt

SEQ ID NO: 39 - DNA, 27 bp, Artificial sequece
  Single strand DNA oligonucleotide
tgcccaatctagtttctgatcttctga

SEQ ID NO: 40 - DNA, 26 bp, Artificial sequece
  Single strand DNA oligonucleotide
aaagacatcaggtccctcccaaagta

SEQ ID NO: 41 - DNA, 25 bp, Artificial sequece
  Single strand DNA oligonucleotide
caatagcagaaacatcccatccaac

SEQ ID NO: 42 - DNA, 27 bp, Artificial sequece
  Single strand DNA oligonucleotide
caatgtttgaaaaggttggtctggact

SEQ ID NO: 43 - DNA, 26 bp, Artificial sequece
  Single strand DNA oligonucleotide
cctcctagccaaacctgatagctgtt

SEQ ID NO: 44 - DNA, 26 bp, Artificial sequece
  Single strand DNA oligonucleotide
tatggtaaaggtggtcagacgagagg

SEQ ID NO: 45 - DNA, 26 bp, Artificial sequece
  Single strand DNA oligonucleotide
gtcatctttttcacgtctagcccaag

SEQ ID NO: 46 - DNA, 27 bp, Artificial sequece
  Single strand DNA oligonucleotide
tgatcaggtcaagaacaatatggagga

SEQ ID NO: 47 - DNA, 29 bp, Artificial sequece
  Single strand DNA oligonucleotide
aagaatcaaacatctgaggaaaagggata

SEQ ID NO: 48 - DNA, 27 bp, Artificial sequece
  Single strand DNA oligonucleotide
gctcgtgtatggtggatgtttagagtc

SEQ ID NO: 49 - DNA, 29 bp, Artificial sequece
  Single strand DNA oligonucleotide
aagttattgggctaattgtttgtccttga

SEQ ID NO: 50 - DNA, 22 bp, Artificial sequece
  Single strand DNA oligonucleotide
ttgtgctggattctggtgatgg

SEQ ID NO: 51 - DNA, 19 bp, Artificial sequece
  Single strand DNA oligonucleotide
ccgctctgctgttgtggtg

SEQ ID NO: 52 - DNA, 26 bp, Artificial sequece
  Single strand DNA oligonucleotide
caggacatcgtgatttcatcaagaac

SEQ ID NO: 53 - DNA, 28 bp, Artificial sequece
  Single strand DNA oligonucleotide
tccatcttgttacaacagcaaatcatct

SEQ ID NO: 54 - DNA, 20 bp, Artificial sequece
  Single strand DNA oligonucleotide
catgccattctccgtcttga

SEQ ID NO: 55 - DNA, 20 bp, Artificial sequece
  Single strand DNA oligonucleotide
cgctcggtcaggatcttcat

SEQ ID NO: 56 - DNA, 25 bp, Artificial sequece
  Single strand DNA oligonucleotide
cctacttgaggctcttgaccagatt

SEQ ID NO: 57 - DNA, 27 bp, Artificial sequece
  Single strand DNA oligonucleotide
aaaagtgacaaccataccaggcttaat

SEQ ID NO: 58 - DNA, 25 bp, Artificial sequece
  Single strand DNA oligonucleotide
cctggaggatgtgagtgttgtaaag

SEQ ID NO: 59 - DNA, 24 bp, Artificial sequece
  Single strand DNA oligonucleotide
agttctctggtatctggtggcttc

SEQ ID NO: 60 - DNA, 27 bp, Artificial sequece
  Single strand DNA oligonucleotide
aagttgtgaaagctcggaatgataact

SEQ ID NO: 61 - DNA, 27 bp, Artificial sequece
  Single strand DNA oligonucleotide
ttctccatcctcatcagatacaacaac

SEQ ID NO: 62 - DNA, 27 bp, Artificial sequece
  Single strand DNA oligonucleotide
tcaagagtatcaggttgcacatattcc

SEQ ID NO: 63 - DNA, 27 bp, Artificial sequece
  Single strand DNA oligonucleotide
ctaaagattcccttcccatcatagaca

SEQ ID NO: 64 - DNA, 27 bp, Artificial sequece
  Single strand DNA oligonucleotide
ttgcacctattacctttctgaccaaat

SEQ ID NO: 65 - DNA, 27 bp, Artificial sequece
  Single strand DNA oligonucleotide
ctcccaggtacatgaccacttcttatt

SEQ ID NO: 66 - DNA, 27 bp, Artificial sequece
  Single strand DNA oligonucleotide
gttgacaaccttatccgtcgattattt

SEQ ID NO: 67 - DNA, 27 bp, Artificial sequece
  Single strand DNA oligonucleotide
gactacagcatcaggttcaaaggattt

SEQ ID NO: 68 - DNA, 1563 bp, Arabidopsis thaliana
ttatgaccttgggatatggtcctgtcatatccacgatggtggatggttcagccactcgtatacctccgtctaccacaaaatccagaccctgtaaaaggaaaccgaaacagagaatagagcaaccaaagatgttctttagtcgctgagaaagagtgagagctataaatacctctggtccatatatgtcacctattgcggttgggtcgatcatccattcattttctttaggccctttcacactgtcattatttaccatacattacacaacttggtcgaagagtccacagtctctctgtagctagtgacgagattttaaagaagaaataaaccttgtgcaaatcaatggggcatccatctgctgcagaatagcttggcagagagcatcatcagatatgcggacacccacgtttttcctagatgcatatttcacacttgtggtgccataacctacacactgtttgggtagttccttgcttgcagtcaagatgaaggtatactgttaaaaaaaaaaaaaatgccaaagatcattgataagaaaactgtttggatattgcgaatatgttgagtatccattagaaaacaagagaagaagaggctctcacaggaccaggtaagcattgcttgacagctcgaaaaacgttagcatgaccatgaccatcaccacgaggaaagcccattgtaaaagtgtcaatatctcgtaaggaacggcataagatgctaagtggctgcataagttgagatggaataatcaaactcaaacctatggaggattcaaattgagcaaagcagaaatatagaacaagtgttccttgcttgcttaccttggaggattcaatctttttaatcctaagaaagagagagagagacacacacgggctaacacgagtcagctcaaaggattggattggaatagaaaagcagcaagagtgagaggtaggtagctaaccttcggagacgctcaacagcggaatggtttttacaatcacaagcaatggcatatctacggagaaacagaaattcaaaaaaaatgcaaaagaggaggaagaagaagaagtaaagaggagaggaagagcttgtttacacggtatcagtgggaataacaccaacagctccttgtttgagaaggtcaatgacaggttggagtttccaggaatcaacaccacaaggatcgacctcgatgtacaccaattccccctccttggtgaaacgaggagtagagtatttgagacgcttagggcttctcttggccgccaacgcgacgatgtatctccgtggagataaagaggcagtgctcggaagcgttgatggtcgccggtgcgtcatcggggaaggaagtagaagaagacgcatcgccgccatacctccgccgtgaagtttggccaccgccatctctcacaatttgtttttgtttgggctcaatttgggctctgtctgttatccacaaattaagagacgcaaacctaaaacgacgccgttccatagttttcctttgaagaagagagagacgtcacaaaggaaagcactcaatcgtcatttcctcggtatctcctcctcgaaagaaga

SEQ ID NO: 69 - DNA, 1562 bp, Arabidopsis thaliana
tgtttgggctcaatttgggctctgtctgttatccacaaattaagagacgcaaacctaaaacgacgccgttccatagttttcctttgaagaagagagagacgtcacaaaggaaagcactcaatcgtcatttcctcggtatctcctcctcgaaagaagaatgcctggaattagaggtccttcggaatactcgcaggaaccacctcgtcacccttctctcaaggtcaacgccaaggaacctttcaacgccgagcctccccgctcggccttagtctcatcttatgtcactcccgtcgaccttttctacaagcgaaatcatggtcccatccccattgttgatcaccttcaaagctactccgtcacccttactggattgatccagaacccgagaaagctctttatcaaagacatcaggtccctcccaaagtacaatgttactgctactcttcagtgtgccggtaacagaaggactgccatgagcaaagttaggaatgttagaggtgttggatgggatgtttctgctattggcaacgctgtctggggtggggcgaaactggccgatgttcttgagcttgtggggataccaaagctgactgcttctaccaatttaggagccagacatgttgagttcgttagtgttgatcgctgtaaggaggaaaatgggggcccttataaggcgtcaatcactctaagtcaagccacaaatcctgaagcggatgttctactcgcttatgagatgaatggagagaccctgaacagggatcacggatttccgttaagggtggttgtccctggtgtgattggtgctcgttcggtcaaatggcttgattccatcaatgtcatcgctgaagaaagccagggattcttcatgcaaaaagattacaaaatgtttccaccctctgtcaattgggataatatcaactggtcctctaggagaccgcaaatggatttccctgttcagagtgcaatctgctctgtggaggatgtgcaaatggtgaagcctggaaaggtaagtatcaaaggatatgcggtttcaggaggtggacgcgggatagaaagagtggacatatccctggatggaggcaaaaactgggtggaagcttctagaacgcaggaaccaggaaagcagtacatctcagaacacagctccagtgacaaatgggcatgggtgttgtttgaagccaccattgatgtttcacagactacagaggtcatcgccaaagcggttgattcggcggcgaatgttcaaccggaaaatgtggagtcggtgtggaacctaagaggggttctcaacacttcgtggcaccgtgtccttctccggcttggccactctaacttgtagaccaagttaccactatgcgtctggtctctctttatcttttctttaagaactaaaacatccttgaatatgcttgtccttgtggatctgtgagagcaaaatactgtttatagttaaataaaatgggaaaacccatctgttgtcatgtggtggattataaattatttatttatatgatagaactgttattcattattatatattaatgaaatcatatggttgagca

SEQ ID NO: 70 - DNA, 1215 bp, Brassica oleracea
atgcctgggattcgcggaccttcagattactcgcaggaaccatctcgtgaccctagtctcaaaatcaacgcaaaggcaagccctcctcctttcttctttgattttgagcccttcaatgccgaaccacctcgctccgccttagtttcatcttacgtcactcccgtccatcttttctacaagcgaaaccatggccccattcccatcatcgatcacatcgacaactactccgtgtccgtcaccggattgatcgataacccaacaaagctcttcatcaaagatatcatgtccctgcccaagtacaatgtcactgctactctacagtgtgcgggtaacagaagaactgctatgagcaaagtcaggaatgttagaggggttggttgggatgtttctgccattggcaacgctgtgtggggcggggccaaattggccgatgttcttgagcttttggggattccaaagctcactagctccaccggtttaggtggcagacatgttgagtttgtcagtgttgatcgctgcaaggaggagaatggtgggccttacaaggcgtcaatccctcttaaccaagccacaaatcctgaagccgatgttctactcgcttatgagatgaatggagagatcctgaacagggatcacggatatccgttaaggctggttgtcccgggtgtgattggtgctcgttccgtcaaatggcttgattccatcaatctactcgctgaagaatgccagggttttttcatgcaaaaagattacaaaatgttccctccttctgtcaattgggacaatatcgactggtcctcaaggaggccccaaatggatttccctgttcagagtgcaatctgctctttggaggacgtgcaaatggtgaagcctggaaaggtaagcatcaaaggatatgcggtttctggaggtgggcgagggatcgagcgagtggatatatcaatggatggaggcaaaagctgggtggaagcttctagaacacagaaaccagggaaggattacatctctgaacacaactccagcgacaaatgggcgtgggtgttgtttgaagccactattgatgtctcccagagtaccaccgaggtcatcgccaaagcggtggattcggcggccaatgttcagccggaaaatgtggagtcagtgtggaacctaagaggggtgctcaatacatcgtggcaccgtgtccttctccgtcttggccactctaacttgtag

SEQ ID NO: 71 - DNA, 1493 bp, Codonopsis lanceolata
gcgacatcgccattgttgcaggacgaagctgaagcaggagaaatgccagggttaagggcgccgtcaaattattcagaagaaccacctcgtcatccttgccttaagatcaatgctaaggaacctttcaatgctgagccacctcgttcagctttaattacctcctacgttacccctgtagatttcttctacaagaggaatcatgggccaatacctgtagttgacgacatagaaaggtattgtttttccataaatgggttaatcgagaaacccaaagagctgtttatgaaagatattcggaatcttccaaaatataatgtaactgctgttttacagtgtgctggtaacagaaggactgccatgagcaaaaccagaacggtaaaaggagttgggtgggatgtttctgctattggaaatgctgtttggggcggggctaaacttgcagatgttcttgaactagttggaataccaaaattaacaagtgtcacaccatggggtggaaaacatgttgaatttgtgagcatcgacaggtgtaaggaggaaaatggagggccttacaaggcatcaatcccattaagtcaagccacaaatcctgaagctgatgttttacttgcatatgagatgaatggagagactctaaacagggatcatggttatccactgcgtgtagtcgttccgggtgttattggtgcccgttctgttaagtggctcgagtccatcaacatagatgccaaagtctgccagggattctttatgcaaaaagattacaaaatgtttccaccttcagttaattgggagaatattgattggtcttccagaaggccacaaatggatttcccagttcagtgtgttatatgctccttggaagacgtaaatgtggtgaagcatggaaaggtagctatcaagggatatgcagtttcaggtggtgggcgtggaattgagagagttgatgtgtctattgatggtggcaaaacctggttggaggcatccagatatcaaaaacccggaattccatacaatgctgatgatgaaagtagcgacaaatgggcatgggtattttttgaggcagaggctgacatacccccaagtgccgaaatagttgctaaagcagtggattcggcagcaaatgtgcaacccgaaaacgttgaagtcatatggaacttgagagggatactgaacacttcatggcatcgtgttcaagtacgagttggtcactcaaacatggattccggatgagaatcttgaaccagaaacgatgcctagctaggcttagagagaaccttggtctaggcattagggttcgaaggagttctttgatcctcacattcagtcgaaagagcttcaaggttgtcaatgggagccgaggttgaagacttgacttgaacccatcgtatgatccactctagtctctactatccaactccggagactggattcttcccaattttatacattcattgctatgtgtagtagcaatctgatttatttattttttc

SEQ ID NO: 72 - DNA, 1539 bp, Solanum tuberosum
cagtgaagccggcggcaaagaaaattgaggaagtgagaaatgcctgggattagagggccttccgattattcgcgagaaccccctcgccatccttgccttaaaattaacgccaaggaaccttttaatgccgagccaactcgttcagctctgatttcatcttatgtcactcctgttgatttcttctacaagagaaaccacggacccataccagtagtggatgacatagagaggtattccgtttctctgagtggtcttattaaaaattccaaagacttgtttatgaaggatatttgtaagcttccaaaatatactgttactgccactttacagtgtgctggtaacagaagaactgctatgagcaagagtcgaacggtgaaaggagttggttgggatattgctgctttaggaaatgctgtttggggtggagcaaaattggcagatgttcttgaattagttggaataccttatttgacaagtatcacacaatctggaggaaaacatgttgaatttgtgagcattgacaagtgtaaggaggaaaatggaggtccttataaggcatcaattccattgagtcaggctacaaaccctgaagctgatgttctgctggcttatgagatgaatggagagccaattaatagggatcatggctatccactgcgtgtggttgtccctggtgtgataggtgcccgttcagttaagtggcttgattacatcaatatcattgccgaagaatgccagggattctttatgcaaaaagactacaagatgtttccaccaacagtgaactgggataacatcaactggtctactaggagaccgcagatggacttccctgttcagagcgcaatatgttccctggaggatgtgagtgttgtaaagcatggaaagataaccatcaaggggtacgcagtatcaggaggtgggcgtggcatagaaagggtagatgtgtctattgatggtggtaaaacttgggaggaagccaccagataccagagaactggcgttccatatattgctgatgattcaagcagtgacagatgggcatgggtcttttttgaagctgaggcaaatattccacagagtgcggaaatagttgctaaagcggtggatatatctgcaaatgtccaacctgaaagtatagattctgtctggaatctgagaggaatcctgaacacctcatggcatcgggttcatgtccgagttggtcaagcaaatctttaggctgtgtttcatgtgttgagcagatgacagtattcagcatcacaggcccaggttgtttgtatcttcctagcttaaaatgaaaaaaacatggttgtttgcatcttcctagcttaaaatgaagatgacattggttgtttgtatcttcctcacttcaaagcaaaaaacactagttgtttgcctcttcctcgacttcaaaagcaagcatgcacatggttgtttgtatcttcttagcttaaaatgaaaatagcatggttgtttgtaacttcctagcttaaaatgaaaatcaaatgttttcttgaaaaaaaaaaaaaaaaaaaa

SEQ ID NO: 73 - DNA, 1548 bp, Oryza sativa
cgtggcaaatccatccatccatcgcctctgcctcgcctccaccaccgagatgccggggctcaccgcgccggcgagctactccgacgagccgcctcgccaccccgccctcaagatcaactccaaggaaccatttaatgctgagcctcatcgatcagcattagtttcatcttatattaccccggtggatttcttctacaagagaaaccatggaccgataccaattgtggatgacctctcaagatacagtgtttccattactggtattgtcaacaagaatgtgcagctgtccatggctgacattaggagtctcccaaagtatgatgtaactgcaacgttacagtgtgcaggcaacaggaggactgcaatgagtaaggtacggaaagtgagaggcgttgggtgggacatatctgctcttggaaatgcaacatggggaggagcaaagctatctgatgtccttgaactagttggaatacctaaactcagctcagtcacatctttaggaggcaagcatgttgagtttgtcagtgttgacaggtgcaaagaagaaaaaggtggtccctataaggcatcaattccactaaagcaggcaacagatcctgaagctgatgtgttacttgcatacgaaatgaacggagagaccctaaaccgtgatcatggatacccacttcgtgttgttgtacctggagtaattggcgcacgctctgtaaaatggcttgacagtatcaacataattgaagaagaatgccagggtttttttgtgcaaaaagattacaagatgtttccaccctccgtagactgggacaatattaattggtcaactaggaggccacaaatggatttccctgtgcagtctgctatttgtaccctggaggatgtggatgttatcaaggaagggaaggctaggattgctggatatgcagtctcaggtggtggccgtggcattgagagagttgatatatctgtcgatggggggaaaacatgggtcgaggctcatagatatcagaaaagcaacgtgccatacatatctgatggacctcggagtgataagtgggcatgggttctattcgaggctacattagacgttccagcaaacgctgagatagtagctaaggcggtggactcagcagcaaatgttcaacctgaaaaggtggaagacgtatggaacctgagaggaatcctcaacacatcttggcatcggatcaaaatacagaattcttcgtgcgtgggaagatccaaaatgtgaaataagtaaactgcttggaatcagaagtcaagagtttatagtgcccggaataataagttattgcagcattcgatgattctcgcagactgtgctcctgtactatagtactatgttggaatcgtgttgctggcctgggcttatatgagacgaaacacaatttttgggagtgatgttaagaagatgattaatacgtactccagtatttatgttgtcttctaatggaaaatttgcagttcttgtgctccatatgataaaagaaaaaactcagtaaattcagttgcaaagatgtgacaagacattaacat

SEQ ID NO: 74 - DNA, 254 bp, Artificial sequence
  AtSO anti sense fragment
ctttctatcccgcgtccacctcctgaaaccgcatatcctttgatacttacctttccaggcttcaccatttgcacatcctccacagagcagattgcactctgaacagggaaatccatttgcggtctcctagaggaccagttgatattatcccaattgacagagggtggaaacattttgtaatctttttgcatgaagaatccctggctttcttcagcgatgacattgatggaatcaagccatttgaccgaacgagc

SEQ ID NO: 75 - DNA, 262 bp, Artificial sequence
  LeSO anti sense fragment
cttacacttgtcaatgctcacaaattccacatgttttcctccagattgtgtgatacttgccaaataaggtattccaactaattctagaacatctgccaatttggctccaccccaaacagcatttcctaaagcagcaatatcccaaccaactcctttcactgttcgactcttactcatagcagttcttctgttaccagcacactgtaaagtggcagtaaccgtatattttggaagcttacaaatatccttcataaacaagtct

SEQ ID NO: 76 - protein, 393 aa, Arabidopsis thaliana
MPGIRGPSEYSQEPPRHPSLKVNAKEPFNAEPPRSALVSSYVTPVDLFYKRNHGPIPIVDHLQSYSVTLTGLIQNPRKLFIKDIRSLPKYNVTATLQCAGNRRTAMSKVRNVRGVGWDVSAIGNAVWGGAKLADVLELVGIPKLTASTNLGARHVEFVSVDRCKEENGGPYKASITLSQATNPEADVLLAYEMNGETLNRDHGFPSRVVVPGVIGARSVKWLDSINVIAEESQGFFMQKDYKMFPPSVNWHNINWSSRRPQMDFPVQSAICSVEDVQMVKPGKVSIKGYAVSGGGRGIERVDISLDGGKNWVEASRTQEPGKQYISEHSSSDKWAWVLFEATIDVSQTTEVIAKAVDSAANVQPENVESVWNLRGVLNTSWHRVLLRLGHSNL

SEQ ID NO: 77 - protein, 404 aa, Brassica oleracea
MPGIRGPSDYSQEPSRDPSLKINAKASPPPFFFDFEPFNAEPPRSALVSSYVTPVHLFYKRNHGPIPIIDHIDNYSVSVTGLIDNPTKLFIKDIMSLPKYNVTATLQCAGNRRTAMSKVRNVRGVGWDVSAIGNAVWGGAKLADVLELLGIPKLTSSTGLGGRHVEFVSVDRCKEENGGPYKASIPLNQATNPEADVLLAYEMNGEILNRDHGYPLRLVVPGVIGARSVKWLDSINLLAEECQGFFMQKDYKMFPPSVNWDNIDWSSRRPQMDFPVQSAICSLEDVQMVKPGKVSIKGYAVSGGGRGIERVDISMDGGKSWVEASRTQKPGKDYISEHNSSDKWAWVLFEATIDVSQSTTEVIAKAVDSAANVQPENVESVWNLRGVLNTSWHRVLLRLGHSNL

SEQ ID NO: 78 - protein, 396 aa, Codonopsis lanceolata
MPGLRAPSNYSEEPPRHPCLKINAKEPFNAEPPRSALITSYVTPVDFFYKRNHGPIPVVDDIERYCFSINGLIEKPKELFMKDIRNLPKYNVTAVLQCAGNRRTAMSKTRTVKGVGWDVSAIGNAVWGGAKLADVLELVGIPKLTSVTPWGGKHVEFVSIDRCKEENGGPYKASIPLSQATNPEADVLLAYEMNGETLNRDHGYPLRVVVPGVIGARSVKWLESINIDAKVCQGFFMQKDYKMFPPSVNWENIDWSSRRPQMDFPVQCVICSLEDVNVVKHGKVAIKGYAVSGGGRGIERVDVSIDGGKTWLEASRYQKPGIPYNADDESSDKWAWVFFEAEADIPPSAEIVAKAVDSAANVQPENVEVIWNLRGILNTSWHRVQVRVGHSNMDSG

SEQ ID NO: 79 - protein, 393 aa, Solanum tuberosum
MPGIRGPSDYSREPPRHPCLKINAKEPFNAEPTRSALISSYVTPVDFFYKRNHGPIPVVDDIERYSVSLSGLIKNSKDLFMKDICKLPKYTVTATLQCAGNRRTAMSKSRTVKGVGWDIAALGNAVWGGAKLADVLELVGIPYLTSITQSGGKHVEFVSIDKCKEENGGPYKASIPLSQATNPEADVLLAYEMNGEPINRDHGYPLRVVVPGVIGARSVKWLDYINIIAEECQGFFMQKDYKMFPPTVNWDNINWSTRRPQMDFPVQSAICSLEDVSVVKHGKITIKGYAVSGGGRGIERVDVSIDGGKTWEEATRYQRTGVPYIADDSSSDRWAWVFFEAEANIPQSAEIVAKAVDISANVQPESIDSVWNLRGILNTSWHRVHVRVGQANL

(19) United States
(12) Patent Application Publication
(10) Pub. No.: US 2011/0265224 A1
(43) Pub. Date: Oct. 27, 2011

(54) CONTROL OF SO2 METABOLISM IN PLANTS AND ITS APPLICATIONS

(75) Inventors: Robert Fluhr, Rechovot (IL); Moshe Sagi, LeHavim (IL)

(73) Assignees: Ben Gurion University of the Negev Research and Development Authority, Beer-Sheva (IL); Yeda Research And Development Co., Ltd., Rehovot (IL)

(21) Appl. No.: 12/449,220

(22) PCT Filed: Jan. 17, 2008
(86) PCT No.: PCT/IL2008/000079

§ 371 Date: Jul. 29, 2009

Related U.S. Application Data
(60) Provisional application No. 60/898,103, filed on Jan. 30, 2007.

(51) Int. Cl. A01H 1/06; A62D 3/02; C12M 1/40; C12N 15/82

(52) U.S. Cl. 800/290; 800/278; 435/468; 435/262.5; 435/283.1

(57) ABSTRACT

SO-transgenic plants overexpressing or lacking SO activity, having modified tolerance or susceptibility to toxicity of sulfite-producing substances, methods for their production and the use thereof for bioremediation of pollutants, as sentinel plants, for enhancing post-harvest quality of plants, plant tissues and plant products and for therapeutic applications.

### FIELD AND BACKGROUND OF THE INVENTION

The present invention relates to transgenic plants expressing exogenous sulfite oxidase (SO) plant materials derived therefrom and uses thereof. More particularly, the present invention relates to SO-modified transgenic plants overexpressing or lacking SO activity, having modified tolerance or susceptibility to toxicity of sulfite-producing substances, methods for their production and the use thereof for bioremediation of pollutants, as sentinel plants or for enhancing post-harvest quality of plants, plant tissues and plant products. The present invention also relates to the use of plant tissues and material having modified levels of SO, and pharmaceutical compositions comprising the same, for therapeutic applications.

Sulfite Oxidase: Sulfite can be oxidized to sulfate by the molybdenum cofactor containing enzyme, sulfite oxidase (SO; EC 1.8.3.1). The enzyme catalyzes a two-electron transfer reaction in which the electrons from sulfite reduce the Moco redox center. The electrons are subsequently transferred to molecular oxygen with simultaneous formation of hydrogen peroxide in addition to sulfate (Eilers et al., 2001; Hansch et al., 2006). Mutations in Moco biosynthetic loci, cnxA-cnxF in Nicotiana plumbaginifolia and nar2a mutant in barley, simultaneously abrogate the activities of SO and the other known plant MoCo-containing enzymes, nitrate reductase (NR; EC 1.6.6.1), xanthine dehydrogenase (XDH; EC 1.1.204) and aldehyde oxidase (AO; EC 1.2.3.1) (Gabard et al., 1988; Muller and Mendel, 1989; Walker-Simmons et al., 1989; Eilers et al., 2001). Specific mutations in the structural genes have also been described for NR that diminish nitrate assimilation (Wilkinson and Crawford, 1993), for XDH that abrogate superoxide production and likely purine catabolism (Yesbergenova et al., 2005), and for AO that diminishes the biosynthesis of the phyto-hormone, ABA (Seo et al., 2000). However, the description of specific SO mutations and their altered expression levels has yet to be reported in plants.

The vertebrate sulfite oxidase is a mitochondrial enzyme containing a heme domain with cytochrome c as the physiological electron acceptor. Human sulfite oxidase deficiency leads to severe neurological abnormalities that often results in death in infancy (Garrett et al., 1998). Among eukaryotes, plant SO is the smallest Mo-containing enzyme known to date and lacks contiguous redox-active centers such as FAD, heme or Fe-S (Eilers et al., 2001). The enzyme is localized in peroxisomes (Eilers et al., 2001; Nowak et al., 2004) and is thus distinct from the multi-enzyme sulfur assimilatory pathway localized to the chloroplast. In sulfur assimilation, plants reduce the ubiquitous sulfate ion through a series of steps that includes activation by ATP sulfurylase and subsequent reduction to the sulfite form by APS reductase (for recent reviews, see (Leustek et al., 2000; Saito, 2004). The sulfite is then reduced by sulfite reductase (SiR; EC 1.8.7.1) by a process that transfers 6 electrons from ferredoxin to produce the fully reduced sulfide form for incorporation into sulfur-containing amino acids (Garsed and Read, 1977; van der Kooij et al., 1997; Saito, 1999, Leustek et al., 2000).

In addition to SO and SiR, additional enzymatic activities in plants are capable of catalyzing sulfite conversion. The UDP-sulfoquinovose synthase 1 (SQD1) protein is localized in the chloroplast and can participate in detoxifying SO₂/sulfite as it catalyzes the transfer of sulfite to UDP-Glc giving rise to UDP-sulfoquinovose (Sanda et al., 2001), an intermediate product for the biosynthesis of sulfolipids needed for proper function of the photosynthetic membranes (Yu et al., 2002, Saito 2004). Additionally, the mitochondrion and cytosol-localized mercaptopyruvate sulfurtransferases, also known as rhodaneses (MST1 and MST2, respectively) have been shown to catalyze the synthesis of the less toxic compound thiosulfate in the presence of 3-mercaptopyruvate and sulfite.

Thus, sulfite can be processed in plants by multiple pathways: in addition to the reductive assimilatory pathway, that takes place in the chloroplast, sulfite can be oxidized to sulfate by SO. As plant SO is a peroxisomal protein presumably peroxisomal catalases can efficiently remove the H202 product (Nowak et al., 2004; Hansch et al., 2006). The nontoxic sulfate product may enter the assimilation pathway or be stored in the vacuole (Kaiser et al., 1989; Leustek and Saito, 1999). The physiological role of SO activity in plants has yet to be established. It has been speculated that SO is required for removing excess sulfite which accumulates upon decomposition of methionine and cysteine or as sulfite arising from other sources such as sulfated metabolites (Heber and Huve, 1998; Hansch and Mendel, 2005).

Plant SO was first cloned and sequenced from Arabidopsis (Eilers et al, JBC 2001; 276:46989-94), revealing a Moco enzyme comprising conserved domains common to other eukaryotic molybdenum binding enzymes, such as sulfite oxidase, nitrate reductase and oxidoreductase. Biochemical characterization of the Arabidopsis enzyme demonstrated similar Km for sulfite, substrate specificity, and sensitivity to inhibition in increased ionic strength, as that of the rat or chicken liver SO (Eilers et al, JBC 2001; 276:46989-94). BLAST analysis reveals that within the plant kingdom, the amino acid sequences of sulfite oxidase from various species such as rice, potato, Codonopsis, and Brassica exhibit up to 87% identity and 92% homology with the Arabidopsis enzyme. Eilers et al further demonstrated antigenic identity of SO proteins from a variety of plant species (tobacco, pea, spinach, barley, carrot, poplar trees and others) with that of the Arabidopsis enzyme. Within the animal kingdom, sulfite oxidase enzymes exhibit sequence identity with the Arabidopsis enzyme of typically less than 50%, and homology of typically less than 65%.

Sulfite-related toxicity: Sulfur dioxide (SO₂) is a gaseous pollutant emitted by natural sources, such as microbial and volcanic activities, and by anthropogenic combustion of sulfur-containing fossil fuels. It is a growing problem in developing industrial countries exacerbated by combustion of ubiquitous sulfur-containing coal, with China leading the world as an SO₂ emitter. In water SO₂ readily hydrates to form the sulfite ions, HSO₃¹⁻ and SO₃²⁻, that are strong nucleophiles that can deleteriously react with a wide variety of cellular components affecting human and plant health. SO₂ enters plants via their stomata and damage is correlated with the degree of stomatal opening (Rennenberg and Herschbach, 1996; van der Kooij et al., 1997). At below toxic levels, plants are able to utilize SO₂. Indeed, sulfur assimilation and biomass production are correlated with SO₂ in the air in sulfate poor soils (Rennenberg, 1984). However, above a certain threshold that differs between plant species, SO₂ toxicity leads to visible effects that include chlorosis (chlorophyll destruction), necrosis (plant tissue death), growth retardation and long term yield reduction (van der Kooij et al., 1997; Noji et al., 2001; Legge and Krupa, 2002).

Debilitation of plants by SO₂ also facilitates pathogen ingress. For example, in a survey covering 160 years, the proliferation of the necrotrophic pathogen Phaeosphaeria nodorum that destroys millions of tons of grain worldwide was shown to correlate with sulfur dioxide pollution in the United Kingdom (Bearchell et al., 2005).

Importantly, and due to its chemical properties, SO₂ and other sulfite-producing substances are routinely used as fumigants, preservatives and food additives for select plants, flowers and plant products such as fresh and dried fruits. Sulfites spontaneously form adducts with many intracellular compounds, including pyrimidine constituents of DNA and are thus antibiotic compounds. Further, the reaction of sulfites with oxygen to form stable SO₄²⁻ produces toxic free radicals. However, limited tolerance of the plants to sulfites, on the one hand, and the potentially toxic effects of ingestion of sulfite-containing material, on the other hand, severely limit the effectiveness of the use of such sulfite-producing substances in agriculture.

Sulfites are known to cause asthma in sensitive individuals, especially severe asthmatics, with a threshold level in the range of three to 130 milligrams of sulfite (SO₂) equivalents. The cause of the reaction is highly sulfited foods, and in extreme cases, the foodborne-induced asthma has resulted in death.

For these reasons, the FDA has banned the use of sulfites on fresh fruits and vegetables served primarily at salad bars, effective August 1986. In April 1990, this sulfite ban was extended to include fresh-peeled potato products, to ensure public safety. Presently, the FDA requires that packaged foods containing 10 ppm sulfite or more must declare this on the label ingredient statement.

Whereas some patents and patent applications have made reference to the hypothetical expression of SO in plants, no SO-modified transgenic plants have been disclosed. Bidney et al (U.S. Pat. Nos. 6,166,291; 6,441,275; 6,376,748; 6,380,460; 6,380,461; and 6,403,861, all incorporated fully herein by reference) teach transgenic plants having enhanced hydrogen peroxide production, the hydrogen peroxide conferring increased resistance to Sclerotina type pathogens. Cahoon et al. (PCT Publication WO0006749, incorporated fully herein by reference) teach the concept, but not the reduction to practice, of transformation of cells with isolated nucleic acids comprising polynucleotides encoding sulfur metabolizing enzymes, such as sulfotransferase and sulfite oxidase, derived from plant cDNA libraries, that may modify sulfur containing components in the cell. The application is one of a large group of applications relating to the hypothetical possibility of producing transgenic plant or animal cells expressing a variety of exogenous genes.

Lalgudi et al (U.S. Pat. No. 6,476,212 and US Patent Application No. 20010051335, both incorporated fully herein by reference) teach the expression of exogenous genes in corn ears and corn tassels. Sulfite oxidase is among the expansive list of potential genes, of plant and animal origin, to be expressed in this system, although no actual reduction to practice or SO-modified transgenic plants or uses thereof for bioremediation or as sentinels are disclosed.

Lang et al (Plant Cell Environ.,2007;30:447-55) have recently reported SO KO Arabidopsis and SO RNAi tobacco plants with no difference in susceptibility to of low levels of SO₂, and enhanced sensitivity of the mutant plants to higher levels of Na bisulfate-generated SO₂. Leaves of a transgenic poplar plant expressing the Arabidopsis SO gene ware reported to have reduced sensitivity to Na bisulfate-generated SO₂. The authors also reported induction of SO enzyme activity and expression in plants exposed to high levels of SO₂.

Thus, although transgenic plants expressing plant sulfite oxidase have been conceived, no bioremediation of sulfate-producing compounds, enhancing nutritional value of the transgenic plants and monitoring levels of sulfite-producing compounds has been disclosed.

Thus, it would be highly advantageous to have methods of modifying tolerance or susceptibility to sulfite producing compounds in plants by expressing exogenous sulfite oxidase, methods for bioremediation of sulfite-producing pollutants, transgenic, hypersensitive plants useful as sentinel plants, or plant tissues and material having modified levels of SO, and pharmaceutical compositions comprising the same, for therapeutic applications, devoid of the above limitations.

### SUMMARY OF THE INVENTION

According to one aspect of the present invention there is provided method of enhancing tolerance of a plant or plant tissue to a sulfite-producing compound, the method comprising expressing an exogenous sulfite oxidase in the plant or plant tissue, thereby enhancing the tolerance of said plant or plant tissue to the sulfite-producing compound.

According to another aspect of the present invention there is provided method of bioremediation of a sulfite-producing compound, the method comprising contacting the compound with at least one transgenic plant expressing exogenous sulfite oxidase, thereby reducing the concentration of said sulfite-producing compound.

According to yet further features in the described preferred embodiments method of claim further comprising the steps of assessing a concentration of said sulfite-producing compound prior to said contacting; and/or assessing a concentration of sulfite-producing compound following said contacting.

According to still another aspect of the present invention there is provided a device for bioremediation of a sulfite-producing compound, the device comprising an at least partially sealed enclosure comprising at least one transgenic plant expressing an exogenous sulfite oxidase, an inlet for directing the sulfite producing compounds to the transgenic plants within said enclosure, thereby remediating the sulfite-producing compounds, and an outlet for removing remediated sulfite-producing compounds from said enclosure.

According to further features in the described preferred embodiments the at least one plant is a plurality of plants.

According to still further features in the described preferred embodiments the sulfite producing compound is selected from the group consisting of sulfur dioxide, sodium sulfite, sodium bisulfite, sodium metabisulfite, sodium dithionite, sulfur, methionine and cysteine, isothiocyanate and isothioyanate glycosides.

According to yet further features in the described preferred embodiments the device further comprising a sensor for measuring a level of a sulfite producing compound.

According to another aspect of the present invention there is provided a method of promoting tolerance to an ingested sulfite-producing compound in a subject in need thereof, the method comprising orally administering to the subject a therapeutically effective amount of an edible plant material derived from a transgenic plant expressing an exogenous sulfite oxidase, thereby promoting tolerance to said ingested compounds in said subject.

According to still another aspect of the present invention there is provided a pharmaceutical composition comprising an edible transgenic plant material expressing an exogenous sulfite oxidase and a pharmaceutically acceptable carrier for oral administration, said transgenic plant having elevated levels of a sulfite oxidase catalytic activity as compared to a similar non-transgenic plant.

According to another aspect of the present invention there is provided a method of enhancing the post harvest quality of a plant or plant tissue in the presence of sulfur compounds, the method comprising upregulating in the plant an activity or level of a sulfite oxidase so as to increase tolerance to sulfur compounds, thereby enhancing post-harvest quality of the plant or plant tissue in the presence of sulfur compounds.

According to still another aspect of the present invention there is provided a method of monitoring levels of sulfite-producing compounds, the method comprising exposing a genetically modified plant having reduced sulfite oxidase catalytic activity as compared to a similar, unmodified plant, to said substance; and monitoring at least one growth parameter of said genetically modified plant, wherein said at least one growth parameter in said plant or portion thereof is reduced by predetermined levels of said sulfite-producing compounds, thereby monitoring levels of sulfite-producing compounds.

According to another aspect of the present invention there is provided an oligonucleotide comprising a nucleic acid sequence capable of specifically hybridizing to a nucleic acid sequence encoding a plant sulfite oxidase and reducing expression of said sulfite oxidase in a plant or plant tissue. Also provided is a nucleic acid construct comprising the nucleic acid sequence and a promoter for directing expression of said nucleic acid sequence in a plant, and a transgenic plant comprising the nucleic acid construct.

According to yet further features in the described preferred embodiments the nucleic acid sequence encoding the plant sulfite oxidase is as set forth in SEQ ID NOs: 16 or 69.

According to still further features in the described preferred embodiments the nucleotide sequence is as set forth in SEQ ID NOs: 75 and 76.

According to yet further features in the described preferred embodiments the oligonucleotide is double stranded.

According to yet further features in the described preferred embodiments the plant comprises an exogenous nucleic acid comprising the sequence as set forth in SEQ ID NOs: 16 and 69-73.

According to yet further features in the described preferred embodiments the plant comprises an exogenous nucleic acid encoding a polypeptide having a sulfite oxidase catalytic activity having an amino acid sequence as set forth by SEQ ID NO: 1 and 76-79.

According to yet further features in the described preferred embodiments the sulfite producing compound is a gas or a liquid.

According to yet further features in the described preferred embodiments the sulfite producing compound is selected from the group consisting of a sulfur dioxide, sulfur, sodium sulfite, sodium bisulfate, sodium metabisulfite, sodium dithionite, methionine, cysteine, isothiocyanate and isothiocyanate glycosides.

According to still further features in the described preferred embodiments the sulfite producing compound is a sulfur dioxide.

According to yet further features in the described preferred embodiments a concentration of said sulfur dioxide is less than or equal to 1 ppm.

According to still further features of the described preferred embodiments a concentration of said sulfur dioxide is 1 to 2 ppm.

According to still further features in the described preferred embodiments a concentration of said sulfur dioxide is greater than 2 ppm.

According to yet further features in the described preferred embodiments the expressing is effected in a tissue specific manner.

According to still further features in the described preferred embodiments the tissue is selected from the group consisting of a leaf, a fruit, a root, a stem and a flower of said plant.

According to yet further features in the described preferred embodiments the plant is selected from the group consisting of plantation plants, orchard plants, field crop plants and ornamental plants.

The present invention successfully addresses the shortcomings of the presently known configurations by providing transgenic plants having modified levels of SO for bioremediation of sulfite-producing pollutants, useful as sentinel plants, and plant tissues and materials having modified levels of SO having enhanced post-harvest quality, and pharmaceutical compositions comprising the same, for therapeutic applications.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-1d are photographs showing the distribution of recombinant SO expression in different tissues and organs in Arabidopsis transgenic plants. Arabidopsis plants were transformed with the β-glucoronidase (GUS) reporter gene (pRITA reporter plasmid, SEQ ID NO: 2) under control of the Arabidopsis SO promoter (SEQ ID NO: 68), designated AtSO::GUS, and the resulting T2 transgenic plants harboring the construct were stained with 5-bromo-4-chloro-3-indoyl-β-D-GluUA for visualization of recombinant expression. FIGS. 1a-1c show staining of different tissues and organs of 3 week old transgenic AtSO::GUS expressing Arabidopsis plants. Note the constitutive expression in all tissues. FIG. 1d is a photograph of a Western blot analysis of SO expression in different plant organs. Plant tissue extracts from transgenic AtSO::GUS and wild type plants were separated on PAGE, immunoblotted with a polyclonal antiserum recognizing a tomato and Arabidopsis synthetic peptide (SEQ ID NO: 27). Immunoreactant bands were visualized with anti-guinea pig anti-IgG (Sigma, Inc, St Louis, Mo.). Each lane contained 50 μg of soluble proteins;

FIGS. 2a-2b are photographs of Western blots showing the modulation of SO expression in transgenic Arabidopsis and Tomato plants. 50 μg of soluble proteins from transgenic Tomato SO overexpressing (OE), Tomato SO RNA interference (Ri) and Arabidopsis SO RNA interference (Ri) lines, and wild type tomato (RR) and Arabidopsis (Col) were fractionated by SDS-PAGE and immunoblotted with Arabidopsis and tomato SO-specific antisera, as described in FIG. 1d. Note the strong (greater than 4 fold) expression of immunoreactive SO protein in the Tomato overexpressing (OE) lines (FIG. 2a, lanes OE12 and OE11), and the absence of SO protein in both the Tomato and Arabidopsis RNA interference (Ri) (SO null) lines (FIG. 2b, Arabidopsis Ri, lanes 5, 8 and 10; and Tomato Ri, lanes 421, 432 and 131). Col=wild-type Columbia Arabidopsis plants, RR=wild type Tomato plants (Lycopersicon esculentum Mill. cv. Rheinlands Ruhm);

FIGS. 3a-3d are histograms and photographs of gels showing the modulation of SO activity in leaf extracts of transgenic Arabidopsis and Tomato plants. SO activity was measured in a kinetic assay (FIGS. 3a and 3c) using the ferricyanide reduction technique (3a and 3c, left inserts) or by following H₂O₂-generation (3a and 3c right insert), in 10 μg of protein extract, and in an SO in-gel assay (FIGS. 3b and 3d). For the in-gel assay, 200 μg of soluble proteins were fractionated in each lane on native-PAGE, and SO activity was visualized with o-dianisidine (3b and 3d, left insert). The bands were excised and refractionated on SDS-PAGE and immunobloted with SO antisera (3b and 3d, right inserts). Note the abundant activity of SO in both assays in the Tomato OE lines (FIGS. 3a and 3b), and the absence of SO activity in both assays in the Tomato (3a and 3b) and Arabidopsis (3c and 3d) SO-null (Ri) lines. Col=wild-type Columbia Arabidopsis plants, RR=wild type Tomato plants (Lycopersicon esculentum Mill. cv. Rheinlands Ruhm);

FIGS. 3e and 3f are histograms showing altered tolerance of SO-modulated plants to SO₂. Sulfate (the product of SO activity) concentration was measured in the leaves of SO₂ treated (+SO₂) and untreated (—SO₂) SO overexpressing (OE) and SO-null (Ri) Tomato and Arabidopsis plants. Leaves were sampled immediately following exposure of the plants to 2 ppm SO₂ for 2 hours. Means±sem (n=4). Two similar experiments in Tomato and Arabidopsis lines yielded essentially identical results. Note the significantly higher sulfate concentrations in the Tomato SO overexpressing lines (FIG. 3e, OE 11), and the lack of increase, relative to the wild type plants, in the Tomato (FIG. 3e, Ri 131), and Arabidopsis (FIG. 3f, Ri 5). Col=wild-type Columbia Arabidopsis plants, RR=wild type Tomato plants (Lycopersicon esculentum Mill. cv. Rheinlands Ruhm);

FIGS. 4a-4b show the effects of modified SO expression on sensitivity to sulfur dioxide in transgenic Arabidopsis SO-null plants. Seven mm diameter leaf discs from 3-week-old WT (control), SO RNA interference (Ri)(Ri10, Ri8, Ri5) lines were placed in 90 mm diameter plates on a filter paper moist with 2 ml of 50% MS salt solution without (control) or with (HSO₃⁻) 7 mM HSO3⁻ for 24 h under constant illumination (100μ einsteins m⁻² s⁻¹) and then were photographed (FIG. 4a), and (FIG. 4b) the remaining chlorophyll content determined. The values of remaining chlorophyll contents after treatment are expressed as the amount of chlorophyll per leaf disc divided by the amount of chlorophyll per untreated control. The “remaining chlorophyll content” in the treated vs. control plants is expressed as a percentage (of untreated control). Note the clear correlation between SO expression and sulfur dioxide sensitivity in the transgenic plants;

FIGS. 5a-5d are photographs of Arabidopsis leaves and a graphic representation of tissue damage showing the effects of modified SO expression on sensitivity to SO₂ pollution. FIG. 5a shows photographs of control (control) and treated (SO₂, 4 h) WT (Col), SO-null (SO RNA interference; RNAi-5, RNAi-8 and RNAi-10) plants 4 days after exposure of plants to 2 ppm SO₂ for 4 hours. FIGS. 5b-5d are graphic representations of the effects of SO₂ on leaf damage level (5b), relative leaf area (5c) and remaining chlorophyll (5d) 4 days after exposure of the transgenic and control plants to 2 ppm SO₂ for 2 hours (left panels) or 4 hours (right panels) as determined 4 days after exposure. Determination of SO₂ effects are expressed as described in detail hereinbelow. Note the consistent correlation between SO₂ toxicity and SO expression in the transgenic plants;

FIGS. 6a-6f are histograms showing the effect of modified SO expression on sensitivity of transcript levels to SO₂ exposure. Relative transcription and protein expression of the senescene marker genes AtWRKY6 (FIG. 6a), ERD/SAG15 (FIG. 6b), ACX1 (FIG. 6c), XERO1/TAS14 (FIG. 6d), ERS/LEA (FIG. 6e), and SRG1 (FIG. 6f) transcript levels in WT(Col) AtSO:35S SO-null (RNA interference) (Ri)(Ri5, Ri8) plants was measured immediately after 2 hours exposure to 2 ppm SO₂ (0 hours, open columns) and 24 hours later (24 h, filled columns) by quantitative RT-PCR, and compared to untreated WT plants after normalization to the Arabidopsis ACT1N2 gene product (At3g18780). The results are mean±SE of three replicates. The data are from one of two different experiments that yielded essentially identical results. Note the hypersensitivity of the SO-null RNAi transformants;

FIGS. 7a-7b are histograms showing the effects of modified SO expression in transgenic Arabidopsis plants, on SO and sulfite reductase expression in response to exposure to SO₂. SO (FIG. 7a) and sulfite reductase (AtSir, FIG. 7b) expression was assessed by quantitative RT-PCR and compared to untreated WT plants [after normalization to the Arabidopsis ACTIN2 gene product (At3g18780)] in wild type (Col) and SO-null AtSO:35s RNAi (Ri 5, Ri8) plants immediately after exposure (0 hours, open columns) or 24 hours (filled columns) after exposure to 2 ppm SO₂ for 2 hours. The results are mean±SE of three replicates. The data are from one of two different experiments that yielded essentially identical results;

FIGS. 7c-7d are histograms showing the effects of modified SO expression in transgenic Tomato plants on SO and sulfite reductase expression in response to exposure to SO₂. SO (FIG. 7c) and sulfite reductase (LeSir, FIG. 7d) expression was assessed by quantitative RT-PCR and compared to untreated WT plants [after normalization to the Tomato actin gene product (Tom41 actin, U60480)] in wild type (RR), SO overexpressing (OE11) and SO-null LeSO:35s RNAi (Ri 131) plants immediately after exposure (0 hours, open columns) or 24 hours (filled columns) after exposure to 2 ppm SO₂ for 2 hours. The results are mean±SE of three replicates. The data are from one of two different experiments that yielded essentially identical results;

FIGS. 8a-8f are histograms illustrating expression of sulfite utilizing genes in transgenic Arabidopsis and Tomato, after exposure to SO₂. Quantitative analysis of MST1 (FIGS. 8a and 8d), MST2 (FIGS. 8b and 8e) and SQD1 (FIGS. 8c and 8f), transcripts by real time RT-PCR was performed using Arabidopsis (FIGS. 8a-8c) and tomato (FIGS. 8d-8f). Arabidopsis and tomato plants were exposed to 2 ppm SO₂ for 2 and 4 hours respectively. Plants used were wild-types (Col and RR), SO RNA interference (Arabidopsis Ri5 and Ri8, tomato R1 131) and tomato overexpression (OE11) plants as indicated and were sampled immediately after SO₂ exposure (0 h) or 24 hours later (24 h). The expressions of each treated line was compared to the untreated line after normalization to the Arabidopsis ACTIN2 gene product (At3g18780) or the tomato actin (Tom41 actin gene, U60480) and displayed as relative expression. Means±s.e.m. (n=3). The data are from one of two different experiments that yielded essentially identical results. Note the elevated level of transcription in the SO-null plants at 24 hours, and not in the wild type or overexpressing plants;

FIGS. 9a-9b are photographs of tomato leaves and a graphic representation of tissue damage showing the effects of modified SO expression on sensitivity to NaHSO₃ in transgenic tomato plants. Nine mm diameter leaf discs from 5-week-old WT tomato SO-null RNA interference (Ri)(Ri432, Ri421, Ri131) and SO overexpressing (OE)(OE11, OE12, OE13) lines were placed on a filter paper moistened with 2 ml of 50% MS salt solution without (control) or with (HSO₃⁻) 7 mM HSO₃⁻ for 24 h under constant illumination (100μ einsteins m⁻² s⁻¹). The discs were photographed (FIG. 9a) and the remaining chlorophyll content (FIG. 9b) in tomato leaf discs were determined, and expressed as percent of untreated control. Note the reduced amount of chlorophyll in SO-null RNA interference but not in overexpressing lines. Chlorophyll is an accepted indication of the health of a plant. Note the consistent correlation between SO₂ toxicity and SO expression in the transgenic plants;

FIGS. 10a-10b are photographs showing impaired tolerance of whole SO-null Arabidopsis plants to SO₂ toxicity. FIG. 10a shows WT (Col), SO RNA interference (Ri 5, Ri8 and Ri10) plants following 2 hours exposure to 2 ppm SO₂ , photographed 4 days later, compared to control untreated plants. FIG. 10b shows control untreated plants versus plants exposed for 4 hours to 2 ppm SO₂, photographed 4 days later. Note the marked, exposure-dependent leaf senescence in all SO-null plants after SO₂ exposure;

FIGS. 11a-11b are photographs showing the altered tolerance of whole transgenic SO-modified tomato plants to SO₂ toxicity. FIG. 11a shows WT (RR), SO-null RNA interference (Ri 131) and SO overexpressing (OE11) following 2 hours exposure to 2 ppm SO₂ , photographed 1 day later, compared to control untreated plants. FIG. 11b is a graph showing the effect of SO₂ treatment on the relative leaf area (left) and measured leaf damage level (right) 1 day after exposure of wild type RR, SO-null (Ri) and SO-overexpressing (OE) tomato plants to 2 ppm SO₂ for 4 hours. Means+s.e.m. (n=4). The data are from one of three different experiments that yielded essentially identical results;

FIGS. 12a-12c are schematic diagrams illustrating the main components of three embodiments of device for bioremediation using SO-modified transgenic plants. FIGS. 12a, 12b and 12c are side views illustrating a first, second and third embodiment of the device, respectively.

FIGS. 13a-13d illustrate the effect of SO₂ fumigation on fungal growth in whole transgenic SO-modified tomato fruit. FIGS. 13a and 13b are photographs of exemplary whole transgenic LeSO overexpressing (OE)(39-LeSO)(13a), transgenic LeSO null (27-LeSO RNAi)(13b) and wild type tomato berries following inoculation with Colletotrichium coccodes hyphal mats. Fruits were exposed to 4 hours at 20 ppm, then 24 hours at 2 ppm SO₂ (+), or no SO₂(−). Note that this regimen of SO₂ exposure arrested fungal growth in both the wild type and transgenic LeSO-OE and LeSO RNAi fruits. FIGS. 13c and 13d are histograms showing that the transgenic LeSO OE(39)(13c) and LeSO RNAi (27)(13d) fruit, as well as the wild type (control) fruit display ripening-stage-dependent sensitivity to SO₂ fumigation.

FIGS. 14a-14c illustrate the effect of SO₂ fumigation on integrity of peduncle and calyx structures in whole transgenic SO-modified tomato fruit. FIGS. 14a and 14b are photographs of exemplary whole transgenic LeSO overexpressing (OE)(39, 4, LE3 and LE5)(14a), transgenic LeSO null RNAi (27, 29 and 2)(14b) and wild type (WT) tomato plants following exposure to 6 hours SO₂ at 2 ppm and 40 hours recovery. While all LeSO OE lines (FIG. 14a) showed greater resistance to SO₂ fumigation, the wild type and LeSO null RNAi lines (FIG. 14b) suffered significant curling of the calyx sepals with fumigation. FIG. 14c shows quantitative assessment of SO₂ damage to the tomato plants, according to the following scale: 5=fully recovered, 4=slightly damaged (beginning of petal curling), 3=medium damage (50% petal curling), 2=damage (75% appear curled and wilted), 1=severe damage (petals and calyx sepals appear wilted). n=7 to 13 fruits.

### DESCRIPTION OF THE PREFERRED EMBODIMENTS

The present invention is of methods for modifying the tolerance or sensitivity to sulfite-producing substances in plants by expressing exogenous sulfite oxidase (SO). Such transgenic plants can be used for bioremediation, to provide a plant-derived source of ingestible SO and to enhance tolerance of plants and their fruit to applications of microcidal sulfur dioxide applications. Also provided are nucleic acid constructs and oligonucleotides for downregulation of SO in the plant tissues, and hypersensitive transgenic plants useful as sentinel plants.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

While conceiving the present invention, it was hypothesized that genetic modification of SO expression in plants might result in altering the plant's capability for metabolism of SO₂ and other sulfite-producing substances.

While reducing the present invention to practice, it was shown, for the first time, that up- or down-regulation of SO expression in plants results in a concomitant alteration in the tolerance of the SO-modified plant to SO₂ and SO₃²⁻. Transgenic plants of the present invention, expressing an exogenous SO gene, exhibit increased SO catalytic activity and can be used to significantly reduce the amount of air- and/or water-borne sulfite-producing pollutants such as SO₂, by enhanced metabolism of the phytotoxic sulfites to sulfate. Furthermore, as demonstrated they remain healthy despite toxic application of sulfur dioxide or sulfite. Similarly, transgenic RNAi SO-null plants have heightened susceptibility to phytotoxicity from SO₂ and other sulfite-producing substances, and can be used to monitor levels of sulfite-producing substances. Additional aspects and applications of the invention are further discussed below.

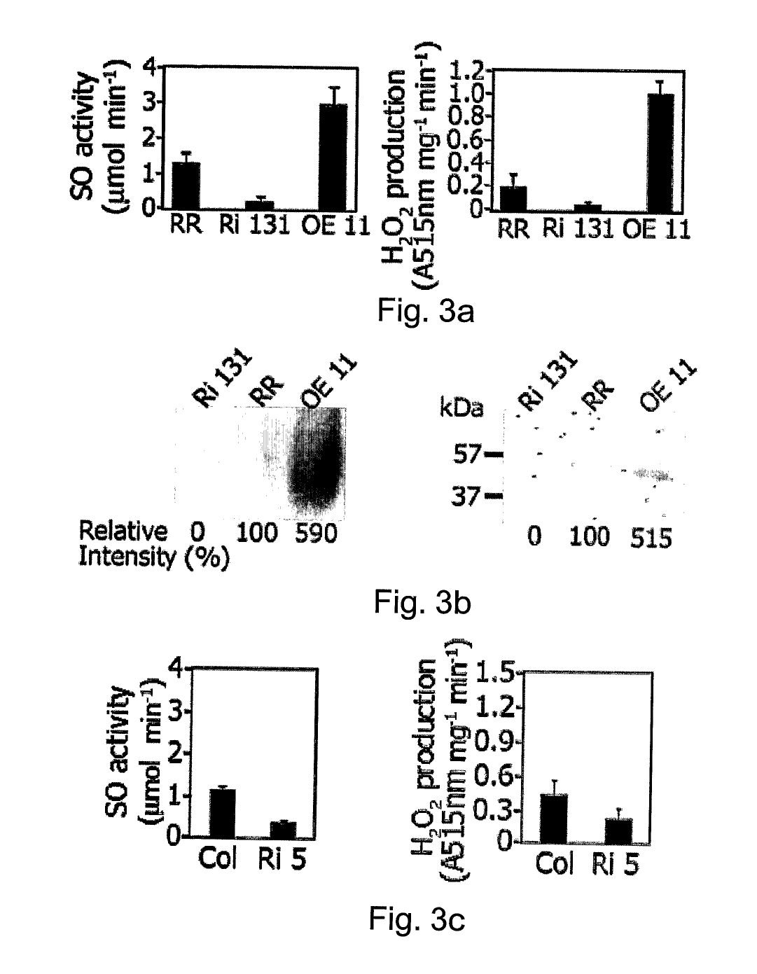

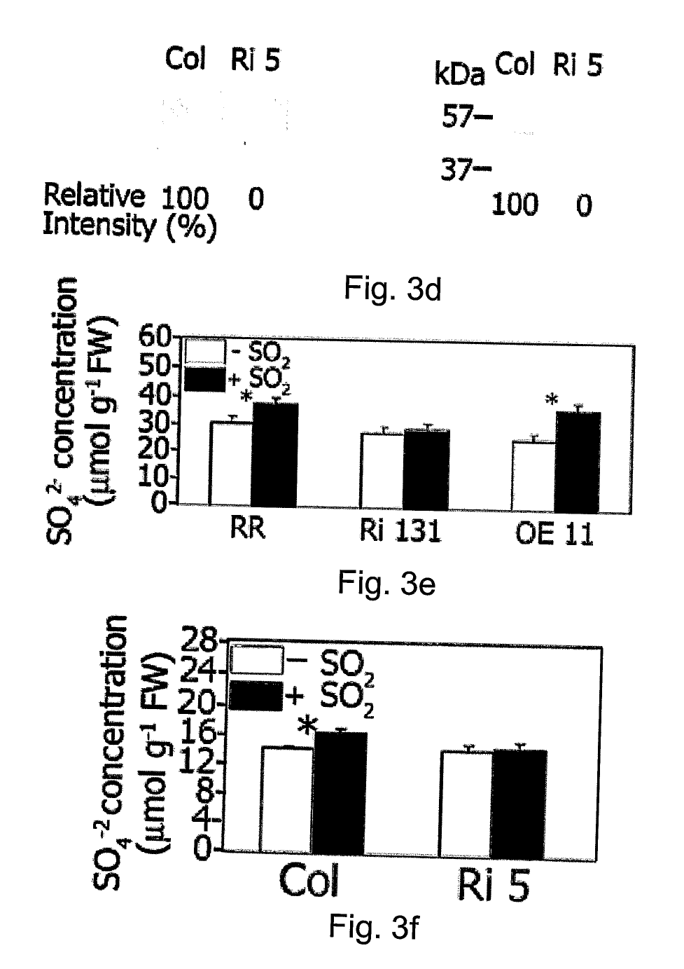

As detailed in Examples 2 and 3 hereinbelow, transformation of tomato plants to overexpress the tomato sulfite oxidase (LeSO) gene resulted in transgenic tomato plants having enhanced SO protein and catalytic activity (FIGS. 2a and 3a) and superior resistance to sulfite toxicity (FIGS. 3e, 9a, 9b and 11a-11b).

Thus, according to one aspect of the present invention, there is provided a method of enhancing the tolerance of a plant to a sulfite-producing compound comprising expressing in the plant an exogenous sulfite oxidase.

As used herein, sulfites (or sulfiting agents) refer to a group of simple chemicals that include sulfur dioxide and sulfite salts, containing the sulfite ion, including synthetic as well as naturally occurring sulfites, found in some foods, mainly those undergoing fermentation.

As used herein, the term “sulfite-producing compound” is defined as any chemical compound which can undergo a chemical reaction to produce the sulfite (SO₃²⁻) ion. Sulfite-producing compounds include, but are not limited to, sulfur dioxide, ionic compounds of the sulfite ion such as sodium sulfite, sodium bisulfite, sodium metabisulfite, sodium dithionite and other sulfur-containing compounds such as sulfur, methionine and cysteine, isothiocyanate and isothioyanate glycosides. The sulfite-producing compound can be in the form of a gas, a liquid, or a solid. For example, SO₂ gas enters plants predominantly via the stomata, where it is metabolized to sulfite, whereas ionic sulfite compounds such as sodium sulfite, in solution, can enter the plant through the leaves, roots and other tissues.

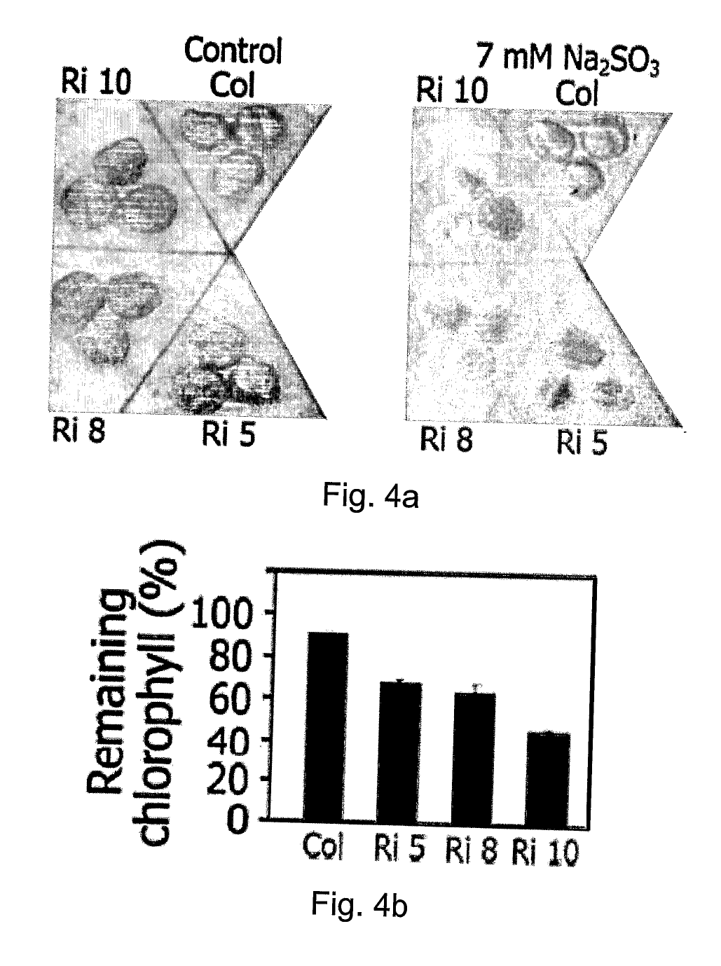

While reducing the present invention to practice, it was uncovered that plant tolerance to sulfite producing substances normally exists up to a threshold, above which phytotoxicity becomes apparent (see FIGS. 4, 5, 10 and 11). Expression of exogenous SO in the transformed plant clearly raises the levels of tolerance to the toxic substance. Thus, in one preferred embodiment, the sulfite producing substance is SO₂, and the concentration, at the plant, is less than 1 ppm, equal to 1 ppm, greater than 1 ppm, or greater than 2 ppm. SO₂ concentration in a gas can be monitored by commercially available, art recognized methods, such as the PM-10 or SO₂ analyzers from Envirotech Instruments, Pvt, LTD (New Delhi, India).

As used herein, the term “plant tolerance” to sulfite-producing compounds is defined as the ability of a plant, or portion thereof, to resist the phytotoxic effects of exposure to sulfite-producing compounds. Such tolerance, and the phytotoxic effects, can be assessed using art-recognized methods. Such methods can include, in a non-limiting manner, parameters such as measurement of biochemical or molecular indicators of stress-related and other enzyme levels (see Examples section hereinbelow), phytomorphic parameters such as leaf area, fruit or flower mass/size or stem height, foliar damage, plant component assessment such as chlorophyll content (see Examples hereinbelow), metabolic parameters such as respiration, photosynthesis, etc., biomass or viability.

The exogenous sulfite oxidase expressed in the transgenic plants of the present invention can be any sulfite oxidase enzyme having sulfite oxidase catalytic activity. It will be appreciated that other enzymes having a sulfite oxidase activity (such as peroxidases) can also be suitable for use with the transgenic plants and methods of the present invention. In a preferred embodiment, the sulfite oxidase is the same sulfite oxidase of the host organism, expressed as an exogenous sulfite oxidase, for example, expression of LeSO in tomato plants. In another embodiment, the exogenous sulfite oxidase is a foreign (heterologous) sulfite oxidase. A non-limiting list of suitable plant sulfite oxidase sequences known in the art includes, for example, sulfite oxidase from A. thaliana (Accession No. AAF13276); B. oleracea (Accession No. ABD65019); C. lanceola (Accession No. BAE48793); S. tuberosum (Accession No. ABB86275) and O. sativa (Accession Nos. BAF24240 and BAF29720). Eilers et al (JBC, 2001, 276:4699-94) have disclosed immunoreactive SO proteins detected in such diverse species as tobacco, pea, spinach, barley, carrot and poplar trees.

In a preferred embodiment, the SO is a polypeptide having a sulfite oxidase catalytic activity. In a more preferred embodiment, the polypeptide having a sulfite oxidase activity is at least 70%, more preferably 75%, yet more preferably 80%, more preferably 85%, more preferably 90%, preferably 95%, and most preferably 100% homologous to SEQ ID NO: 1. Assays for determining sulfite oxidase activity are well known in the art e.g., the ferricyanide reduction assay and the o-dianisidine “in-gel” assay described hereinbelow.

As mentioned hereinabove, SO is ubiquitous in the plant kingdom. Thus, almost all plants are suitable for use with the present invention. Suitable plants include dicots and monocots. Specifically, the transgenic plant(s) of the invention can be selected from the non-limiting list of orchard plants such as apples, avocado and pears, orange, grapefruit, lemon, persimmon, peach, plum, apricot, cherry, nectarine, almond, pecan, walnut, and filbert; plantation plants such as banana, oil palm, olive, Douglas fir, teak; field crop plants such as alfalfa, rice, wheat, barley, rye, cotton, sunflower, peanut, corn, potato, sweet potato, bean, pea, chicory, lettuce, endive, cabbage, brussel sprout, beet, parsnip, turnip, cauliflower, broccoli, turnip, radish, spinach, onion, garlic, eggplant, pepper, celery, carrot, squash, pumpkin, zucchini, cucumber, apple, pear, melon, citrus, strawberry, grape, raspberry, pineapple, soybean, tobacco, tomato, sorghum, papaya, and sugarcane; and suitable ornamental plants such as Rosa, Arabidopsis thaliana, Saintpaulia, petunia, pelargonium, poinsettia, chrysanthemum, carnation, and zinnia.

It will be appreciated that upregulation, or downregulation of SO expression can be performed using whole plants, parts thereof, cells thereof, and plant cells in culture. Thus, SO-overexpressing or SO-null plants of the present invention, methods for their use, and pharmaceutical compositions thereof, can be plant tissues such as fruit, and plant cells in culture as well as whole plants.

In order to generate transgenic plants a nucleic acid sequence encoding sulfite oxidase (described hereinabove) is ligated into a nucleic acid construct suitable for plant expression. It will be appreciated that the nucleic acid sequence encoding SO can be expressed in organisms outside of the plant kingdom, such as prokaryotes, yeast, algae, and other non-plant eukaryotes, using methods of transformation and recombinant expression well known in the art.

The transgenic plants of the present invention can be transformed by stable or transient transformation. In stable transformation, a nucleic acid molecule capable of up-regulating SO expression is integrated into the plant genome and as such it represents a stable and inherited trait. In transient transformation, the nucleic acid molecule is expressed by the cell transformed but it is not integrated into the genome and as such it represents a transient trait.

In a preferred embodiment, the nucleic acid molecule comprises a sequence encoding a polypeptide having a sulfite oxidase catalytic activity. The nucleic acid molecule can be 70%, more preferably 75%, yet more preferably 80%, more preferably 85%, more preferably 90%, preferably 95%, and most preferably 100% homologous to SEQ ID NO: 16. In another embodiment, the nucleic acid comprises the SO coding sequence from other plants, as set forth in SEQ ID NOs. 69-73.

There are various methods of introducing foreign genes into both monocotyledonous and dicotyledonous plants (Potrykus, I., Annu. Rev, Plant, Physiol., Plant. Mol. Biol. (1991) 42:205-225; Shimamoto et al., Nature (1989) 338:274-276).

The principle methods of causing stable integration of exogenous DNA into plant genomic DNA include two main approaches:

(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.

(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

In a preferred embodiment, DNA is directly transferred into the plant cell. There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

In another embodiment, the transgenic plant is transformed by the Agrobacterium system. The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledenous plants.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Although stable transformation is presently preferred, transient transformation of leaf cells, meristematic cells or the whole plant is also envisaged by the present invention.

Transient transformation can be effected by any of the direct DNA transfer methods described above or by viral infection using modified plant viruses.

Viruses that have been shown to be useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (By), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of the present invention is demonstrated by the above references as well as in U.S. Pat. No. 5,316,931, incorporated fully by reference herein.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that said sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

In addition to the above, the nucleic acid molecule of the present invention can also be introduced into a chloroplast genome thereby enabling chloroplast expression.

A technique for introducing exogenous nucleic acid sequences to the genome of the chloroplasts is known. This technique involves the following procedures. First, plant cells are chemically treated so as to reduce the number of chloroplasts per cell to about one. Then, the exogenous nucleic acid is introduced via particle bombardment into the cells with the aim of introducing at least one exogenous nucleic acid molecule into the chloroplasts. The exogenous nucleic acid is selected such that it is integratable into the chloroplast's genome via homologous recombination which is readily effected by enzymes inherent to the chloroplast. To this end, the exogenous nucleic acid includes, in addition to a gene of interest, at least one nucleic acid stretch which is derived from the chloroplast's genome. In addition, the exogenous nucleic acid includes a selectable marker, which serves by sequential selection procedures to ascertain that all or substantially all of the copies of the chloroplast genomes following such selection will include the exogenous nucleic acid. Further details relating to this technique are found in U.S. Pat. Nos. 4,945,050; and 5,693,507 which are incorporated herein by reference. A polypeptide can thus be produced by the protein expression system of the chloroplast and become integrated into the chloroplasts inner membrane.

According to a preferred embodiment the nucleic acid construct according to this aspect of the present invention further comprises ^{a} promoter for regulating exogenous SO expression in a sense orientation. Such promoters are known to be cis-acting sequence elements required for transcription as they serve to bind DNA dependent RNA polymerase which transcribes sequences present downstream thereof. While the exogenous SO-encoding polynucleotide described herein is an essential element of the invention, it can be used in different contexts. The promoter of choice that is used for expression of the exogenous SO is of secondary importance, and will comprise any suitable promoter. It will be appreciated by one skilled in the art, however, that it is necessary to make sure that the transcription start site(s) will be located upstream of an open reading frame. In a preferred embodiment of the present invention, the promoter that is selected comprises an element that is active in the plant host cells of interest. In a yet more preferred embodiment of the present invention, the promoter further comprises a sulfite sensitive regulatory element (e.g. enhancer element), for inducing high levels of SO expression in the presence of SO and/or a SO-producing compound.

In many instances it is desired to target the expression of an exogenous recombinant protein. Such targeting can be into a cellular organelle or outside of the cell. This can be affected, as is well known in the art, by appropriate signal peptides, which are fused to the polypeptide to be targeted, typically at the N terminus, such as the ER retention signals KDEL, HDEL and the like.

The phrase “signal peptide” refers to a stretch of amino acids which is effective in targeting a protein expressed in a cell into a target location. Different signal peptides, which are known in the art, are effective in secreting a protein from bacteria, yeast, plant and animal cells.

It should be noted in this respect that signal peptides serve the function of translocation of produced protein across the endoplasmic reticulum membrane. Similarly, transmembrane segments halt translocation and provide anchoring of the protein to the plasma membrane (see, for example, Johnson et al. The Plant Cell (1990) 2:525-532; Sauer et al. EMBO J. (1990) 9:3045-3050; Mueckler et al. Science (1985) 229:941-945). Mitochondrial, nuclear, chloroplast, or vacuolar signals target expressed protein correctly into the corresponding organelle through the secretory pathway (see, for example, Von Heijne, Eur. J. Biochem. (1983) 133:17-21; Yon Heijne, J. Mol. Biol. (1986) 189:239-242; Iturriaga et al. The Plant Cell (1989) 1:381-390; McKnight et al., Nucl. Acid Res. (1990) 18:4939-4943; Matsuoka and Nakamura, Proc. Natl. Acad. Sci. USA (1991) 88:834-838; Cunningham and Porter (Recombinant proteins from plants, Eds. C. Cunningham and A. J. R. Porter, 1998 Humana Press Totowa, N.J.).

Endogenous SO expression in plants is normally restricted to the peroxisome. Thus, in one preferred embodiment, the exogenous SO polypeptide of the SO-modified transgenic plant is targeted to the peroxisome. Methods and specific sequences, such as the PST1 and PST2 sequences for targeting of recombinant proteins to peroxisomes are described in Johnson et al (Plant Physiol. 2001; 127:731-39; and U.S. Pat. Nos. 6,103,956 and 6,914,170 to Srienc et al and Li et al., respectively, which are incorporated herein by reference).

It will be appreciated that, depending on the ambient conditions, and the type of sulfite-producing compounds to which the plant is exposed, it could be advantageous to direct expression of the exogenous SO to a specific portion or organ of the plant. Thus, in one embodiment of the invention, SO expression levels are genetically modified in a differential manner, in specific tissues or portions of the plant, such as the leaves, fruit, root, stem, flower, etc. Such organ-specific expression in plants can be directed using organ, and stage-specific promoters, such as the light and vasculature specific promoters disclosed by Coruzzi et al. (U.S. Pat. No. 5,391,725 incorporated herein by reference), the temporal- and organ-specific promoters taught by Meier et al (Plant Physiol. 1995;107:1105-18, incorporated herein by reference) or the ethylene-inducible promoters taught by Sessa et al (Plant Mol Biol, 1995;28:145-53, incorporated herein by reference).

It will be appreciated that plants or crop plants and their wild relatives can be screened to identify individual plants, cultivars or species having increased SO expression levels, as a result of natural variation. Such screening can be based, for example, on detection, in plants, of levels of SO transcription, abundance of SO enzyme protein and/or SO catalytic activity, or identification of plants having a sulfite-resistant phenotype, using methods well known in the art. Selected plants can then be cultivated by traditional breeding techniques to produce progeny having enhanced SO levels, and the modified tolerance to sulfite producing substances resulting therefrom. Further, plants having suitably enhanced SO levels screened, identified, selected and propagated by the methods described herein can be used in any of the therapeutic applications disclosed herein.

Methods of assaying SO catalytic activity include, but are not limited to the ferricyanide reduction assay as described hereinbelow, and as described in Eilers et al (JBC 2001;276:46989-94). Briefly, the reduction of ferricyanide in the presence of SO and sulfite is monitored spectrophotometrically at 420 nm.

Methods of detecting the SO polypeptide include, but are not limited to imunodetection using a specific anti-SO antibody (see Examples hereinbelow), purification on ion-exchange followed by size exclusion chromatography as described by Eilers et al (JBC 2001;276:46989-94); by quantitative immunodetection and separation on gel electrophoresis and immunodetection as described in detail hereinbelow.

Methods for detecting expression of SO in plants also include, but are not limited to the assay of transcription of the SO gene, by PCR, as described hereinbelow. For example, transcript levels can be estimated by extension of a homlogous ologonucleotide probe, by fractionation of RNA on agarose gels followed by detection in the Northern blot method or in a similar manner by dot blot.

Sulfite levels can be determined by extraction in and then applying a colorimetric pararosaniline/formaldehyde assay modified by Zhao, et al (Drug Metab. Dispos., 1999;27,992-998.} or by its enzymatic conversion to sulfate as described in the methods below.

SO₂ can be assayed, for example, by any of following assays: the Monier-Williams (M-W) distillation assay, high performance liquid chromatography (HPLC); aeration-oxidation method; and rapid distillation followed by redox titration, as described in detail by Williams et al. (Am. J. Enol and Vitic 1992, 43:227-29).

Sulfate levels can be accurately determined by direct ion exchange chromatography system as described in the methods below.

The methods of the present invention can be used to produce plants expressing an exogenous SO having enhanced tolerance to sulfite-producing compounds, which can be used to provide pollution-resistant trees, shrubs, crops, flowers, etc. for regions in which sulfite-producing pollution, such as SO₂, is above phytotoxic threshold levels. For example, urban and industrial regions could benefit from hardier, less care-intensive vegetation by cultivating the transgenic SO-modified plants. Further, crop yields and cost-effectiveness of farming in regions having high levels of sulfite-producing air- or water pollutants could be improved by planting transgenic SO-modified crops having enhanced tolerance to the pollutants.

It will be appreciated, that while sulfite-related phytotoxicity may not always kill an affected plant, exposure to such compounds results in a weakened, and subsequently disease- and pathogen-prone plant. Thus, another advantage of the use of the transgenic plants expressing the sulfite oxidase of the present invention is a greater resistance of the genetically modified plant(s) to non-SO-related disease and pathogens.

Herbicide or fumigant resistance in plants is desirable due to a number of advantages: (i) increased options—the availability of an extra herbicide option is extremely valuable. In some cases it simply allows the crop to be grown and in others it provides an alternate mode of action for the management of herbicide resistance development in the weed population; (ii) more flexibility—the increased options provide greater flexibility in terms of crop rotations and the ability to respond quickly to market opportunities; (iii) increased safety—where safer chemicals are able to be used, the risks to personal safety are clearly reduced. There may also be advantages where the chemical involved is environmentally benign or is used in very low concentrations.[see, for example, glyphosate (Roundup™) and glufosinate (Basta™)]

Plants of the present invention having enhanced tolerance to sulfur compounds can withstand the use of said sulfur compounds, or of higher concentrations of said compounds, as agents in weed, fungus and/or pest control. The use of such agents can be coordinated with the growth of specific crops, for example, those crops being specially sensitive to fungal or microbacterial damage. Specific herbicides having a known target of action, such as glyphosate, and modified crop plants having enhanced tolerance thereto, have been marketed together for improved crop yield. Thus, plants expressing modified levels of SO, and having enhanced tolerance to sulfur-containing compounds, can also be provided in combination with sulfur-containing herbicides, fumigants and pesticides.

While reducing the present invention to practice, it was surprisingly uncovered that sulfate concentration in the plant following exposure to sulfite producing pollutants such as SO₂ is directly correlated to the levels of SO expression (see Example 2, and FIGS. 3a-3b hereinbelow). Thus, transgenic plants having enhanced tolerance to sulfite producing pollutants are able to take up, and metabolize greater amounts of such pollutants than similar, non-modified plants.

Thus, the present invention can provide a method of bioremediation of a substance comprising a sulfite-producing compound, the method comprising contacting a substance comprising said compound with a transgenic plant genetically modified to express elevated levels of sulfite oxidase, thereby reducing the concentration of the sulfite-producing compound(s) in the substance.

Bioremediation exploits the capacity of living organism to remove toxic compounds from contaminated air, water or soils. In the case of plants (phytoremediation), applications include, for example, the removal of pollutants such as heavy metals and/or organic pollutants by plant species that are able to concentrate the pollutants at higher levels than those found in the soil or water (“hyperaccumulators”) and/or preferably metabolize the pollutants to less toxic compounds (for a recent review of the subject, see Peuke et al. EMBO 2005;6:497-501). Methods for bioremediation of airborne, water-borne and soil pollutants using native and genetically engineered vegetation have been described in detail in the art (see Weeks et al, U.S. Pat. No. 7,022,896; Alberte et al, U.S. Pat. No. 6,841,718; and Austin, U.S. Pat. No. 7,087,169, all incorporated herein by reference). However, no methods for phytoremediation of sulfite-producing compounds have been disclosed.

Thus, according to another aspect of the present invention there is provided a method of bioremediation of a sulfite-producing compound, the method comprising contacting the compound with at least one transgenic plant expressing exogenous sulfite oxidase, thereby reducing the concentration of said sulfite-producing compound. Methods for bioremediation of sulfite-producing compounds according to the present invention include, but are not limited to, the use of transgenic plants expressing exogenous SO of the present invention to “scrub” sulfite producing compounds (i.e. SO₂) from ambient air, the use of such plants to remove sulfite producing compounds from waste water, contaminated water, etc., and use of transgenic plants expressing exogenous SO of the invention for remediation of contaminated soil (for example, removal of residual fertilizers from soil). According to one aspect of the invention, the contaminated or polluted substance to be remediated can be a gas, a liquid or a solid (such as soil, etc.). It will be appreciated that bioremediation of a substance containing sulfite-producing compounds can be affected by passive contact of the sulfite producing compounds with the transgenic plants, dependent on natural movements of air, water and soil, or, according to another embodiment, the substance for bioremediation can be actively directed to produce contact with the transgenic plants.

In one preferred embodiment, the substance is a gas, and the sulfite-producing compound is SO₂. Bioremediation according to the present invention can be effected using a single SO-modified plant, a few plants, or a plurality of plants comprising large numbers of bioremediating plants. Bioremediation can be effected by SO-modified plants selected from, but not limited to, any of plants that are suited for indoor or outdoor use. A non-limiting list of indoor plants includes Spathiphylulum (peace lily) Dracaena species (Janet Craig), Boston fern, Chrysanthemum, Gerber daisy, Dwarf date palm and Bamboo palm.

For outdoor use, as in cities as landscape plants or in the case of industry, suitable plants are, for example, trees or shrubs belonging to pinus, eucalyptus, picea, populus, quercus and acacia, or areas planted with combinations of trees and shrubs.

While reducing the present invention to practice, the sulfite-remediating capacity of a transgenic plant expressing exogenous SO was assessed. The plants were placed in a closed cabinet in air that was supplemented with 2 ppm SO₂, exceeding the 1 ppm toxicity threshold for humans. Since sulfite is found in low levels in plant tissues, and is difficult to recover and measure, the sulfate (SO product) concentration was monitored instead. Following 2 hours exposure, the plants were processed for sulfate content. It was found that provided with identical conditions, the transgenic plants expressing exogenous SO convert, on an average, 20% more sulfite than untreated controls, and 30-40% more sulfite than similar, SO-null SO-antisense plants, converting the toxic sulfite into easily metabolized, harmless sulfate (SO₄). Thus, the bioremediation capacity of the transgenic plants expressing exogenous SO is considerably enhanced.

Calculating the bioremediation potential of the SO-modified plants is done as follows. When tomato plants with enhanced SO are exposed to 2 ppm of sulfur dioxide there is a 10 micromolar increase in sulfate measured in plant tissue after 2 hours. Thus, the transgenic, SO overproducing plants bioremediate sulfur dioxide into sulfate at approximately the rate of 5 sulfate/gm fresh weight/hour. As the sulfite (SO₃²) originates from SO₂, there is an equimolar relationship. At STP, 1 mole of gas occupies 24.46 liters, thus 1 micromole of gas occupies 24.46 microliters volume. Thus, 5×10⁻⁶ mole (5 micromole)×24.46 liters=122.3 microliters of SO₂ per hour per 1 gram fresh weight. Thus, 1 gram fresh weight of plant material is capable of “scrubbing” 122.3 microliters, or the equivalent of 122 ppm SO₂ per hour per liter of air.

It will be appreciated, that the capability for bioremediation of SO₂ from the surrounding medium depends on the sensitivity or tolerance of the plants to the phytotoxic effects of the SO₂, since damaged plants lose metabolic robustness. Thus, the transgenic plants expressing exogenous SO, as described herein have enhanced tolerance to sulfite, and are best suited for use in bioremediation of sulfite-producing compounds.

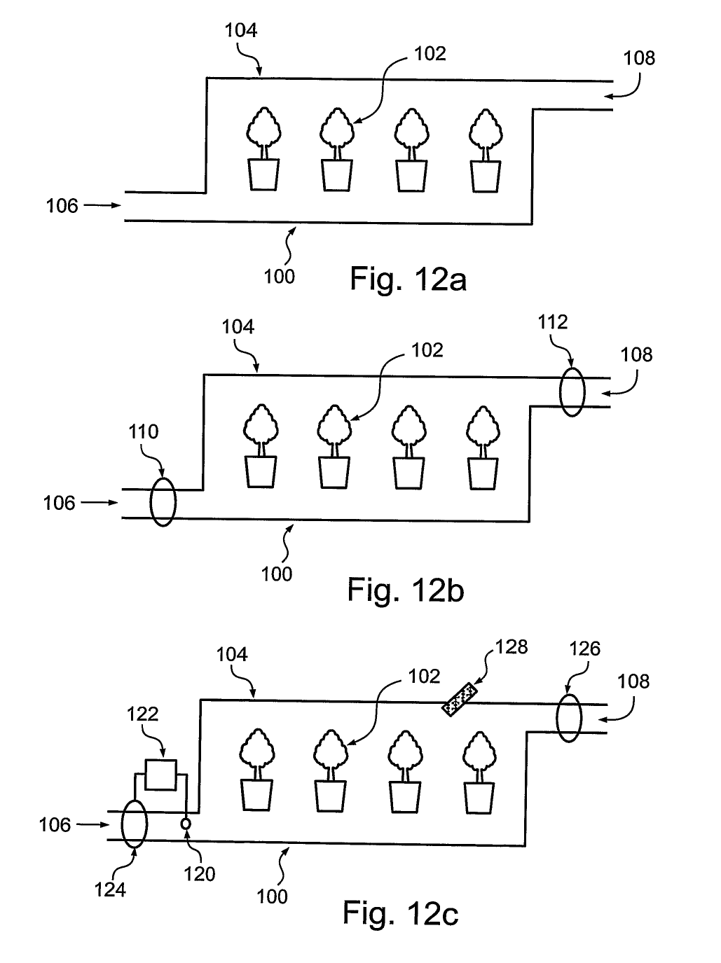

The methods for bioremediation of the present invention can be used in urban environments or around industrial area in natural settings such as tree or shrub landscape planting or adapted for use in a device for bioremediation of sulfite-producing compounds. Such a device comprises an enclosure comprising transgenic plants expressing elevated levels of SO for exposing the plants to a sulfite-producing substance. In a preferred embodiment, the bioremediation device further comprises one or more components for active introduction (e.g. a pump) of the substance to be remediated, at least one component for the active removal thereof following exposure to the SO-modified plant(s). The substance can be a gas, a liquid or a solid. FIG. 12a is a cross-sectional schematic illustrating one such bioremediation device (100), having transgenic SO-modified plants (102) enclosed in a simple box-like enclosure (104), having inlet (106) for introducing the substance to be remediated, and outlet 108 for removal of the treated substance. In the case of a gas, the inlet can be in functional association with a component for actively introducing the substance into the enclosure 110, such as a fan, impeller, blower, pump or the like. Outlet 108 can optionally be in functional association with a similar component (112) for active removal of the remediated substance from the enclosure (see FIG. 12b). Similar means for introduction and removal of liquids and solids to and from the enclosure can be envisaged. Further devices suitable for bioremediation and methods for their use have been previously described (see, for example, U.S. Pat. Nos. 6,869,539 to Sheets for devices for biological water decontamination and U.S. Pat. No. 5,635,394 to Horn for devices and methods for air bioremediation, both of which are incorporated fully by reference). Such water containing devices would include specific expression enhanced of SO in root or in aquatic plants such as duckweed for use in water bioremediation.

Additional embodiments of the present invention include methods and devices for bioremediation of sulfite producing compounds, further comprising assessing the level of sulfite-producing pollutant or contaminant before contact with the transgenic plant expressing exogenous SO of the invention and assessing sulfite producing compounds following exposure to the plants, in order to monitor the efficacy of bioremediation. FIG. 12c is a cross sectional schematic illustrating such a device, having a sensor for monitoring the levels of sulfite producing compounds (120) in functional association with a controller (122), for regulating the activity of a pump (124) located at the inlet (106), so as to modulate the rate of inflow and amount of contact between the sulfite producing compounds and the plants. In another embodiment, the sensor can modulate the rate of outflow via a suction pump (126) at the outlet (108). SO₂ can be assayed, for example, by any of following assays: the Monier-Williams (M-W) distillation assay, high performance liquid chromatography (HPLC); aeration-oxidation method; and rapid distillation followed by redox titration, as described in detail by Williams et al. (Am. J. Enol and Vitic 1992, 43:227-29). Further embodiments can include device having a sensor (128) to monitor plant vigor (e.g. a color sensor for monitoring the extent of chlorosis) for indicating overexposure of the plant to sulfite or sulfite producing compounds.

The operation of a device as described herein would be understood by one of ordinary skill in the art. Briefly, at least one transgenic plant (102) expressing exogenous SO is provided within the enclosure (104), and a substance for bioremediation comprising a sulfite- or sulfite-producing compound is introduced through the inlet 106, so as to contact the plant. Flow of the substance for bioremediation from the inlet, via the plant, and out through the outlet can be via suction at the outlet or by pressure at the inlet, or passive.

It will be appreciated that aquatic plants, such as algae and higher aquatic species, are suited for bioremediation of sulfites and sulfite-producing compounds dissolved in liquids, and can be used in the device described herein.

As mentioned in the “Background” section hereinabove, sulfur compounds, such as SO₂ and H₂S are commonly used for fumigation and pest control in flowers and fruit. One widespread use of such compounds, resulting from their effective elimination of microorganisms, is their application following harvesting, in order to enhance shelf life, and storage capability, transport range and the quality of the produce/flowers reaching market. However, levels of sulfur compounds effective in such fumigation are limited by the plants' own limited tolerance to sulfate (see, for example, Czapskil et al. J. Food Sci. 2000;65:722, relating to storage of mushrooms; and H M Mustonen Aust. J. Exp. Agric. 1992;32:389-393, relating to storage of grapes). For example, Crisosto et al. (Int. Symp. on Table Grape Prod., 1994; pg 195-199, Am. Soc for Enology and Vinoc.) reported that while the use of SO₂ fumigation and SO₂ box liner pads effectively reduced stem browning and botrytis in table grapes, extended exposure to effective concentrations of SO₂ also impaired the post-harvest quality of the grapes. Grapes, or other fruit having enhanced tolerance to sulfite producing compounds such as SO₂ would be better able to withstand higher concentrations of sulfur-containing fumigants.

While reducing the present invention to practice, it has been shown that transgenic plants modified to express increased levels of sulfite oxidase have enhanced tolerance to fumigation with sulfur compounds such as sulfur dioxide (see Examples 2 and 3 below). Thus, according to one aspect of the present invention there is provided a method of producing plants having enhanced post harvest quality in the presence of sulfur compounds, comprising expressing in the plants an exogenous sulfite oxidase, so as to produce transgenic plants expressing increased levels of sulfite oxidase having enhanced post harvest quality in the presence of sulfur compounds. Post-harvest quality can be expressed in terms of length of shelf life, storage capability, transport range, and can be determined by such parameters as stem and fruit browning, botrytis inoculation level, anthracnose infections, wilting, and the like. Expression of the exogenous sulfite oxidase can be in the entire plant, as shown in Examples 2 and 3 herein, or can be targeted and restricted to a commercially desirable portion of the plant or plant tissue, such as flowers, fruit, leaves, roots, etc, using well known methods of recombinant gene expression, as detailed herein. It will be appreciated that screening methods for identifying plants having enhanced SO expression can be employed to identify naturally occurring cultivars capable of producing plants or plant tissues having such enhanced post harvest quality.

As described herein, sensitivity to inhaled or ingested sulfur dioxide or sulfite or sulfite-producing compounds is a well known and widespread phenomenon. In addition to the toxic effects, ingestion of sulfite-producing compounds often induces asthma in sensitive individuals. Where sulfites or sulfite-producing compounds such as SO₂ are used in food or drinks, for example, as a preservative, such sensitive individuals must either abstain from their ingestion, or chance an adverse reaction.

Increasing the amount of sulfite oxidase in the diet of a sensitive individual could effectively reduce the sensitivity to ingested sulfites or sulfite-producing compounds. Thus, the transgenic plants expressing exogenous SO and having enhanced levels of SO of the present invention can be used to produce a therapeutic, plant-derived sulfite oxidase. Administration of a therapeutic amount of a plant-derived SO to a sulfite-sensitive subject can thus be used to promote tolerance to ingested sulfite-producing compounds in the subject.

Thus according to yet another aspect the administration of the plant derived SO is via ingesting or inhalation of a plant material derived from a transgenic SO-modified plant having enhanced SO activity.

Thus, plant and plant parts including stems, fruits, flowers or roots of the transgenic plants expressing exogenous SO and having increased SO expression, described herein, can be used to counteract the effects of SO₂ fumigation by enhanced tolerance, on the one hand, and can further be administered to produce enhanced tolerance when ingested themselves. Administration can be via pre-treating the food with formulated SO extracted from a over-producing source to accelerate detoxification before ingestion. Administration can be via ingestion of the transgenic plant expressing exogenous SO, or of plant-derived SO following extraction of an active fraction from the plant or plant material. Or by treating the food from a source enriched with SO extracted from a plant. It will be appreciated that the source of SO for such a use can also be a preparation containing SO derived from other, non-plant sources such as microorganisms. Any art-recognized methods for preparation of plant materials and extraction of the SO fraction can be used herewith, providing that the exogenous SO of the plant materials or extract is active and free of counterindicated material.

Thus, according to another aspect of the present invention there is provided a pharmaceutical composition comprising a transgenic plant or transgenic plant material expressing exogenous SO, the plant or plant material having elevated levels of SO catalytic activity as compared with a control, non-transgenic plant, and a physiological carrier. It will be appreciated that the suitability of plants or plant cells having elevated levels of SO activity for use in such pharmaceutical compositions will be dependent on their compatibility with the metabolism and physiology of the intended subject, e.g. lack of toxicity, absence of serious side effects, etc, that can be determined empirically, as described hereinbelow.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the SO accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal or intestinal delivery.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by oral or nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (sulfite oxidase) effective to prevent, alleviate or ameliorate symptoms of a disorder (e.g., allergic asthma) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (see e.g., Fingl, et al., 1975, in “The Pharmacological Basis of Therapeutics”, Ch. 1 p. 1, incorporated herein by reference).

Dosage amount and interval may be adjusted individually to provide plasma or brain levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

Compositions of the present invention may, if desired, also be presented as a food additive, or a medicinal food.

Enhanced sulfite oxidase activity in plants can produce a downstream effect of increasing biosynthesis of essential thiol-containing amino acids, such as methionine and cysteine. Thus, according to another aspect of the present invention there is provided a method of enhancing the nutritional value of a plant or plant product comprising upregulating in the plant an activity or level of a sulfite oxidase, producing an increased sulfur amino acid content in the plant. Plants or plant products having increased sulfur amino acid content would be advantageous for use in animal feed, and for human nutrition.

As used herein, the term “plant product” is defined as the plant, a portion thereof, or a plant-derived material, processed or packaged so as to retain the enhanced SO activity characteristic of the whole transgenic plant.

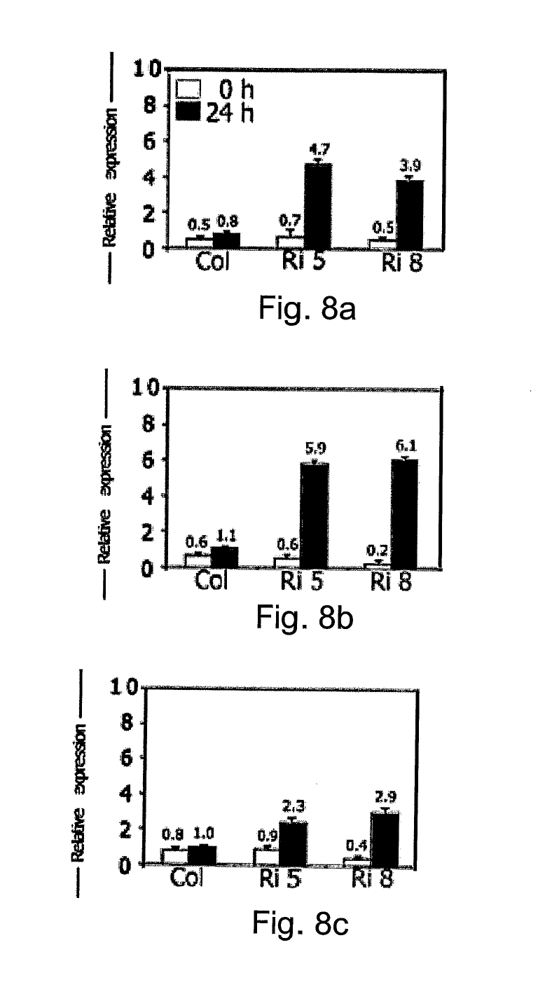

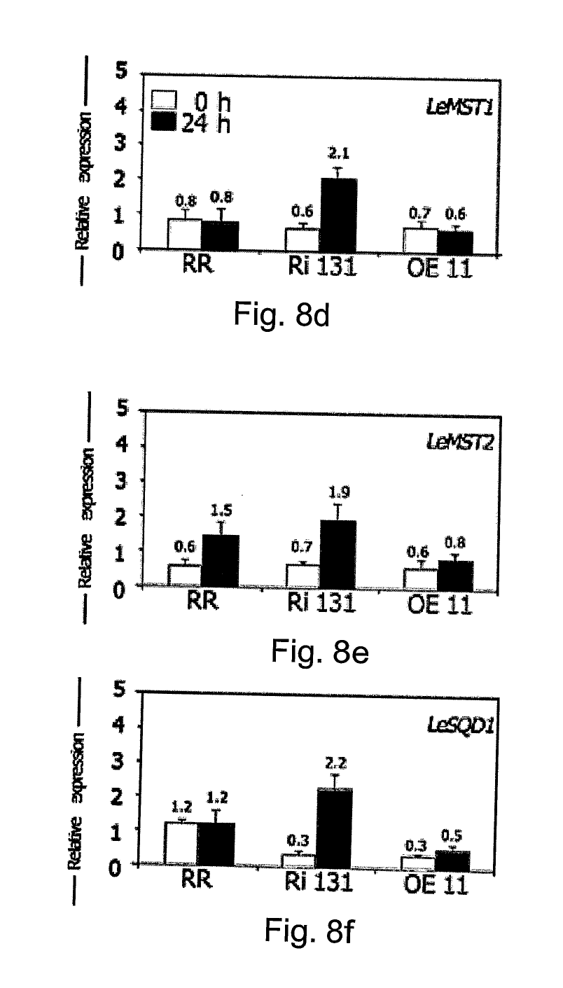

While reducing the present invention to practice, it was postulated that reducing the levels of SO in transgenic plants would result in transgenic plants having reduced tolerance (e.g. heightened sensitivity and susceptibility) to sulfites and sulfite-producing compounds. SO-null plants having reduced expression of SO were produced by transforming plants with specific SO antisense constructs (see Examples 2 and 3 hereinbelow). After determining that the SO-null plants lack immunologically detected SO polypeptide (FIGS. 2 and 8), it was shown that exposure of SO-null anti-SO antisense plants to SO₂ and Na₂SO₃ resulted in greater sulfite-related toxicity than in wild type or SO-enhanced plants (FIGS. 4a-4b, 5a-5d, 6a-6f, 7a-7d, 9a-9b, 10a-10b and 11a-11b).

Thus, transgenic plants genetically modified to have reduced expression of SO of the present invention, having reduced sulfite oxidase catalytic activity as compared to a similar, unmodified plant, can be used for monitoring levels of a sulfite-producing compound in a substance. Monitoring levels of the sulfite-producing compounds is effected by exposing the transgenic genetically modified plant to the substance in question, and monitoring at least one growth parameter indicative of sulfite-related toxicity in the plant.

The use of such sentinel plants, having enhanced susceptibility to the detrimental effects of a compound or substance of interest, is well known in the art. Harper et al (U.S. Pat. No. 7,109,033) disclosed the use of transgenic plants over-expressing stress-related genes, and their detection by exposure to typical damage-type stress. In crops, sentinels having reduced SO levels can be dispersed among a field of similar, but non-modified plants, and observed to signal increased levels of sulfite-producing pollutants, by the character and/or extent of morphological or biochemical sulfite-related damage to the plant. Indicative parameters useful in the present invention include, but are not limited to leaf chlorophyll content, morphological (size, wet weight, etc), metabolic, biochemical and molecular criteria, as detailed hereinbelow. Further, solitary plants having reduced SO levels can be maintained in mobile containers, placed in contact with air, water or soil suspected of having sulfite-related contaminants, and observed to detect concentrations approaching toxic levels thereof. In another embodiment, plant health parameters indicative of sulfite-related toxicity can be further monitored in the sulfite-sentinel plants by a remote and/or automated system for assessment of levels sulfite-producing compounds.

Thus, there is provided an oligonucleotide comprising a nucleic acid sequence capable of specifically hybridizing with a nucleic acid sequence encoding a plant sulfite oxidase, thereby reducing expression of the sulfite oxidase in a plant or plant tissue, a nucleic acid construct for the expression thereof in a plant, and a transgenic plant transformed with the nucleotide construct, having reduced sulfite oxidase catalytic activity.

Downregulation of SO in the plants can be effected on the genomic and/or the transcript level using a variety of molecules which interfere with transcription and/or translation (e.g., antisense, siRNA, Ribozyme, DNAzyme). Following is a list of agents capable of downregulating expression level and/or activity of SO in plants.

One agent capable of downregulating a SO activity is a small interfering RNA (siRNA) molecule. RNA interference is a two step process. The first step, which is termed as the initiation step, input dsRNA is digested into 21-23 nucleotide (nt) small interfering RNAs (siRNA), probably by the action of Dicer, a member of the RNase III family of dsRNA-specific ribonucleases, which processes (cleaves) dsRNA (introduced directly or via a transgene or a virus) in an ATP-dependent manner. Successive cleavage events degrade the RNA to 19-21 bp duplexes (siRNA), each with 2-nucleotide 3′ overhangs [Hutvagner and Zamore Curr. Opin. Genetics and Development 12:225-232 (2002); and Bernstein Nature 409:363-366 (2001)].

In the effector step, the siRNA duplexes bind to a nuclease complex to from the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore Curr. Opin. Genetics and Development 12:225-232 (2002); Hammond et al. (2001) Nat. Rev. Gen. 2:110-119 (2001); and Sharp Genes. Dev. 15:485-90 (2001)]. Although the mechanism of cleavage is still to be elucidated, research indicates that each RISC contains a single siRNA and an RNase [Hutvagner and Zamore Curr. Opin. Genetics and Development 12:225-232 (2002)].

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC [Hammond et al. Nat. Rev. Gen. 2:110-119 (2001), Sharp Genes. Dev. 15:485-90 (2001); Hutvagner and Zamore Curr. Opin. Genetics and Development 12:225-232 (2002)]. For more information on RNAi see the following reviews Tuschl ChemBiochem. 2:239-245 (2001); Cullen Nat. Immunol. 3:597-599 (2002); and Brantl Biochem. Biophys. Act. 1575:15-25 (2002).

Synthesis of SO RNAi molecules is described in detail hereinbelow (see Examples, Materials and Methods section). In general, synthesis of RNAi molecules suitable for use with the present invention can be effected as follows. First, the SO mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene. Another agent capable of down-regulating SO is the use of tailored microRNA vectors. In this case specific sequences for SO are embedded within a pre-set microRNA inducing vector. This vector is then attached to a specific promoter to achieve tissue specific down-regulation.

Another agent capable of downregulating SO expression is a DNAzyme molecule capable of specifically cleaving an mRNA transcript or DNA sequence of the SO. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al., which is incorporated herein by reference. DNAzymes of similar design directed against the human Urokinase receptor were recently observed to inhibit Urokinase receptor expression, and successfully inhibit colon cancer cell metastasis in vivo (Itoh et al, 20002, Abstract 409, Ann Meeting Am Soc Gen Ther www.asgt.org). In another application, DNAzymes complementary to bcr-ab1 oncogenes were successful in inhibiting the oncogenes expression in leukemia cells, and lessening relapse rates in autologous bone marrow transplant in cases of CML and ALL.

Downregulation of a SO can also be effected by using an antisense polynucleotide capable of specifically hybridizing with an mRNA transcript encoding the SO.

Design of antisense molecules which can be used to efficiently downregulate a SO must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16: 1374-1375 (1998)].

For example, suitable antisense oligonucleotides targeted against the SO mRNA (which codes for the SO protein) are described in the Examples section hereinbelow (see Example 2)

Several clinical trials have demonstrated safety, feasibility and activity of antisense oligonucleotides. For example, antisense oligonucleotides suitable for the treatment of cancer have been successfully used [Holmund et al., Curr Opin Mol Ther 1:372-85 (1999)], while treatment of hematological malignancies via antisense oligonucleotides targeting c-myb gene, p53 and Bcl-2 had entered clinical trials and had been shown to be tolerated by patients [Gerwitz Curr Opin Mol Ther 1:297-306 (1999)].

More recently, antisense-mediated suppression of human heparanase gene expression has been reported to inhibit pleural dissemination of human cancer cells in a mouse model [Uno et al., Cancer Res 61:7855-60 (2001)].

Effective antisense-mediated suppression of a variety of plant genes has been reported, for example, maize DIMBOA biosynthesis genes (U.S. Pat. No. 6,331,660), histone deacylase genes (U.S. Pat. No. 6,808,926), tobacco nicotine synthesis genes (U.S. Pat. No. 5,668,295) and tomato polygalacturonase genes (U.S. Pat. No. 5,669,831), all of which are incorporated herein by reference.

Thus, the current consensus is that recent developments in the field of antisense technology which, as described above, have led to the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Another agent capable of downregulating a SO is a ribozyme molecule capable of specifically cleaving an mRNA transcript encoding a SO. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

An additional method of regulating the expression of an SO gene in cells is via triplex forming oligonuclotides (TFOs). Recent studies have shown that TFOs can be designed which can recognize and bind to polypurine/polypirimidine regions in double-stranded helical DNA in a sequence-specific manner. These recognition rules are outlined by Maher III, L. J., et al., Science, 1989; 245:725-730; Moser, H. E., et al., Science, 1987; 238:645-630; Beal, P. A., et al, Science, 1992; 251:1360-1363; Cooney, M., et al., Science, 1988; 241:456-459; and Hogan, M. E., et al., EP Publication 375408. Modification of the oligonuclotides, such as the introduction of intercalators and backbone substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer, J Clin Invest 2003; 112:487-94).

In general, the triplex-forming oligonucleotide has the sequence correspondence:

| | | | | |
|---|---|---|---|---|
| oligo | 3′--A | G | G | T |
| duplex | 5′--A | G | C | T |
| duplex | 3′--T | C | G | A |

However, it has been shown that the A-AT and G-GC triplets have the greatest triple helical stability (Reither and Jeltsch, BMC Biochem, 2002, Sep. 12, Epub). The same authors have demonstrated that TFOs designed according to the A-AT and G-GC rule do not form non-specific triplexes, indicating that the triplex formation is indeed sequence specific.

Thus for any given sequence in the SO regulatory region a triplex forming sequence may be devised. Triplex-forming oligonucleotides preferably are at least 15, more preferably 25, still more preferably 30 or more nucleotides in length, up to 50 or 100 bp.

Transfection of cells (for example, via Agrobacterium) with TFOs, and formation of the triple helical structure with the target DNA induces steric and functional changes, blocking transcription initiation and elongation, allowing the introduction of desired sequence changes in the endogenous DNA and resulting in the specific downregulation of gene expression. Examples of such suppression of gene expression in cells treated with TFOs include knockout of episomal supFG1 and endogenous HPRT genes in mammalian cells (Vasquez et al., Nucl Acids Res. 1999; 27:1176-81, and Puri, et al, J Biol Chem, 2001; 276:28991-98), and the sequence- and target specific downregulation of expression of the Ets2 transcription factor, important in prostate cancer etiology (Carbone, et al, Nucl Acid Res. 2003; 31:833-43), and the pro-inflammatory ICAM-1 gene (Besch et al, J Biol Chem, 2002; 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res 2000; 28:2369-74).

Additionally, TFOs designed according to the abovementioned principles can induce directed mutagenesis capable of effecting DNA repair, thus providing both downregulation and upregulation of expression of endogenous genes (Seidman and Glazer, J Clin Invest 2003; 112:487-94). Detailed description of the design, synthesis and administration of effective TFOs can be found in U.S. Patent Application Nos. 2003 017068 and 2003 0096980 to Froehler et al, and 2002 0128218 and 2002 0123476 to Emanuele et al, and U.S. Pat. No. 5,721,138 to Lawn.

If desired, the level of SO transcripts, or protein or enzyme activity may be assessed to determine if the desired change (up- or down-regulation) in SO expression has been achieved. Methods for assessing transcripts, proteins and SO are known in the art. As detailed hereinbelow, synthesis and level of SO transcripts may be assessed by, for example, northern blot analysis or RNase protection assays, and SO protein may be determined by, for example, western blotting. SO activity may also be determined by enzyme activity assays.

Flatulence having unpleasant odor following ingestion of sulfur-rich foods, such as legumes, is the result of enteral bacterial action on the thiol-containing amino acids. This is a benign yet socially significant problem in most developed countries. Reduction of the sulfur content of plant-derived foods can be beneficial in reducing the odiferous flatulence potential of the foods. Thus, according to another aspect of the present invention there is provided a method of producing a plant or plant product having reduced odiferous flatulence potential, the method comprising reducing the sulfur content of the plant by downregulating SO activity in the plant, so as to decrease the concentration of sulfur containing amino acids in the plant. Downregulating SO activity can be affected by expressing in the plant an oligonucleotide comprising a nucleic acid sequence capable of specifically hybridizing with a nucleic acid sequence encoding a plant sulfite oxidase, thereby reducing expression of the sulfite oxidase in the plant or plant tissue, as detailed hereinabove.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorpotaed by reference as if fully set forth herein. Other general references are provided throughout this document. The procedures therein are believed to be well known in the art and are provided for the convenience of the reader. All the information contained therein is incorporated herein by reference.

### Materials and Experimental Methods

### Materials and Experimental Methods

Plant materials and growth conditions: A. thaliana plants (ecotype Columbia) were grown in 50% Murashige and Skoog (MS) agar plates or trays containing low-nutrient soil in growth room at 8 h light/16 h darkness, 22° C., 75-85% relative humidity, and 100 μeinsteins m⁻² s⁻¹. Tomato (Lycopersicon esculentum Mill. cv. Rheinlands Ruhm) plants were grown in the growth room in pots filled with a peat and vermiculite (4:1 v/v) mixture containing slow-release High N multicote 4 with micro-elements (Haifa Chemicals Ltd, Israel; 0.3% w/w).

Preparation of RNA: For quantitative reverse transcriptase-polymerase chain reaction (RT-PCR) and AtSO gene silencing, total RNA was prepared by using the Aurum™ total RNA Mini Kit (BIO-RAD, Hercules, Calif.) according to the manufacturer's instructions. For cloning of tomato SO (LeSO)(GenBank Accession No:DQ853413) cDNA, total RNA was extracted with the RNeasy plant mini kit (Qiagen, Valencia, Calif.).

AtSO Genes Silencing Lines

AtSO gene silencing: 0.5 μg of Arabidopsis leaf total RNA were reverse-transcribed with an iScript™ cDNA Synthesis Kit using modified MMLV-derived reverse transcriptase (BIO-RAD, Hercules, Calif.) and a blend of oligo-d(T) and random hexamer primers according to the manufacturer's instructions. The resulting cDNA was used as template for isolating a 254-bp-length PCR product (SEQ ID NO: 74). The fragment was introduced as sense and antisense in pRNA69 plasmid containing a 35S promoter of cauliflower mosaic virus upstream to the sense and antisense multi-cloning site separated by a 631-bp-length intron. The forward primer was 5′ CGGGATCCCTCGAGGCTCGTTCGGTCAAAT 3′ (433-447 bp in the SO gene) (SEQ ID NO: 3) containing BamHI and XhoI restriction sites (in bold), and the reverse primer was 5′ CCATCGATGAATTCCTTTCTATCCCGCGTCCA 3′ 667-685 bp in the SO gene) (SEQ ID NO: 4) containing ClaI and EcoRI sites (in bold). The sense fragment was ligated to pRNA69 plasmid via the restriction sites XhoI and EcoRI and then the antisense fragment was ligated to the plasmid through the restriction sites BamHI and ClaI. The resulting construct was digested with NotI and a fragment containing the 35S promoter and the inserted AtSO fragments were ligated to the NotI site in the binary vector pML-BART. The construct was introduced into Agrobacterium tumefaciens strain GV3101 by electroporation transformed to Columbia plants using a floral dip method (Clough and Bent, 1998). Transformed lines were selected by resistance to Basta® (Glufosinate ammonium; Aventis CropScience, Victoria, Au). For AtSO interference lines verification, the antisense specific fragment was amplified using the primer 5′ GGGCTTTGACATCTTTGAAGAAAAC 3′ (528-556 bp in the SO gene) (SEQ ID NO: 5) that span the intron region of the pRNA69 plasmid and 5′ TCAATTGGGATAATATCAACTGGTCCTC 3′ (SEQ ID NO: 6) as reverse primer. The sense specific fragment was amplified using the reverse primer 5′ AAAACTTACATTCTTGGCAGCAGTG 3′ (SEQ ID NO: 7) that span the intron region of the pRNA69 plasmid and 5′ TCAATTGGGATAATATCAACTGGTCCTC 3′ (528-556 bp in the SO gene) (SEQ ID NO: 8) as forward primer. Genomic DNA extracted from resulting transgenic plants resistant to Basta® was employed as template. For transgene verification genomic DNA was examined for the presence of the 255 and 202-bp-length PCR products flanking the prokaryotic intron and the antisense and sense cDNA inserts, respectively and were separated on a 2% agarose is gel, excised from the gel and sequenced.

Plants Harboring AtSO: GUS Construct and Histochemical GUS Staining

AtSO promoter analysis: The Arabidopsis BAC F1C9 (Accession No. AC011664; obtained from the Arabidopsis Biological Research Center, Colombus, Ohio) was used as template. The forward primer was 5′ AGACTCGAGTATGACCTT GGGATATGGTCCTGTC 3′ (905000-90525 bp in the BAC F1C9 sequence) (SEQ ID NO: 9) containing XhoI restriction site (in bold), and the reverse primer was 5′ TCCAAGCTTTCTTCTTTCGAGGAGGAGATACCGAG 3′ (92036-92062 bp in the BAC F1C9 sequence) (SEQ ID NO: 10) containing HindIII site (in bold). The resulting PCR product of 1562-bp-length was ligated to pRITA plasmid, via the restriction sites XhoI and HindIII. The resulting construct was digested with NotI and a fragment containing the inserted AtSO promoter upstream to b-glucuronidase (GUS) reporter gene, was ligated to the NotI site in the binary vector pML-BART. The resulting construct was introduced into Agrobacterium tumefaciens strain GV3101 by electroporation, transformed to Arabidopsis (ecotype Columbia) plants using a floral dip method (Clough and Bent, 1998).

For construct verification, a specific fragment was amplified using the primer 5′ AGGAAACAGCTATGACCATGATTACGA 3′ (SEQ ID NO: 11) that spans the multi-cloning site region of the pRITA plasmid and reverse primer 5′ TTTGTGGTAGACGGAGGTATACGAGTG 3′ (90551-90577 bp in the BAC F1C9 sequence) (SEQ ID NO: 12) of the promoter insert. The 189-bp-length PCR product was separated on a 2% agarose gel, excised from the gel and sequenced for verification. The T2 plants harboring the constructs were stained using 5-bromo-4-chloro-3-indolyl-β-D-GlcUA.

Cloning of tomato SO cDNA and generation of tomato RNAi and overexpression lines: Total RNA (1.5 mg) was subjected to first-strand synthesis using SuperScript II reverse transcriptase (Gibco BRL, Cleveland, Ohio) according to the manufacturer's procedure with GeneRacer™ (Invitrogen, Carlsbad Calif.) oligo-d(T) primer GCTGTCAACGATACGCTACGTAACGGCATGACAGTG(T)₂₄(SEQ ID NO: 13). PCR amplification was conducted on one-tenth of the reaction using the following primers: forward 5′ CAAGTCACACAGCACCGTTT 3′ (001-020 bp in the LeSO gene) (SEQ ID NO: 14) and reverse 5′ GCTGTCAACGATACGCTACGTAACG 3′ (GeneRacer™, Invitrogen, Carlsbad Calif.) (SEQ ID NO: 15), resulting in a 1581-bp full length LeSO cDNA product. The obtained cDNA of LeSO (SEQ ID NO: 16) (GenBank Accession NO. DQ853413) was directly ligated to pGEM-T Easy (Promega, Madison, USA) and sequenced.

For LeSO gene silencing, the resulting full length LeSO cDNA (SEQ ID NO: 16) was used as template for isolating a 262-bp-length PCR product (SEQ ID NO: 75). The fragment was introduced into pRNA69 plasmid as described for AtSO gene silencing using forward primer 5′ CGGGATCCCTCGAGAGACTTGTTTATGAAG 3′ (405-421 bp in the LeSO gene) (SEQ ID NO: 17) containing BamHI and XhoI restriction sites (in bold) and reverse primer 5′ CCATCGATGAATTCCTTACACTTGTCAATGCT 3′ (649-667 bp in the LeSO gene) (SEQ ID NO: 18) containing ClaI and EcoRI sites (in bold). The resulting construct was digested with NotI and a fragment containing the 35S promoter and the inserted LeSO fragments were ligated into the Nod site in the binary vector pART27. The construct was introduced into Agrobacterium tuinefaciens strain GV3101 by electroporation transformed to tomato plants (Lycopersicon esculentum Mill. cv. Rheinlands Ruhm). Stable transformation was performed with Agrobacterium-mediated transformation according to McCormick (1991).

For LeSO interference lines verification the antisense specific fragment was amplified using the primer 5′ GGGCTTTGACATCTTTGAAGAAAAC 3′ (SEQ ID NO: 19) that span the intron region of the pRNA69 plasmid and 5′ GGATATTGCTGCTTTAGGAAATGCTGT 3′ (525-552 bp in the LeSO gene) (SEQ ID NO: 20) as reverse primer. The sense specific fragment was amplified using the reverse primer 5′ AAAACTTACATTCTTGGCAGCAGTG 3′ (SEQ ID NO: 21) that span the intron region of the pRNA69 plasmid and 5′ GGATATTGCTGCTTTAGGAAATGCTGT 3′ (525-552 bp in the LeSO gene) (SEQ ID NO: 22) as forward primer. Genomic DNA extracted from resulting transgenic plants resistant to kanamycin was employed as template. The 240 and 187-bp-length PCR products containing partial prokaryotic intron and the antisense and sense cDNA inserts, respectively were separated on a 2% agarose gel, excised from the gel and sequenced for verification as described.

For LeSO overexpression, the resulting full length LeSO cDNA was used as template for isolating an 1182-bp-full length LeSO cDNA. The fragment was introduced first in pART7 plasmid using forward primer for LeSO over expression constructs 5′ ACACTCGAGATGCCTGGGATTAAAGGGCC 3′ (175-195 bp in the LeSO gene) (SEQ ID NO: 23) containing XhoI restriction site (in bold) and reverse primer 5′ TACGAATTCCTAAAGATTTGCTTGACCAAC 3′ (1336-1357 bp in the LeSO gene) (SEQ ID NO: 24) containing EcoRI sites (in bold). The resulting construct was digested and ligated to the NotI site in the binary vector pART27, introduced into Agrobacterium tumefaciens strain GV3101 and transformed to tomato plants (Lycopersicon esculentum Mill. cv. Rheinlands Ruhm), as described. For verification, specific fragment was amplified using the primer 5′ ATCATTGCGATAAAGGAAAGGCTATCA 3′ (SEQ ID NO: 25) that spans the multi-cloning site region of the pART7 plasmid and reverse primer 5′ GAATAATCGGAAGGCCCTTTAATCC 3′ (182-207 bp in the LeSO gene) (SEQ ID NO: 26) of the cDNA insert. Genomic DNA extracted from resulting transgenic plants resistant to kanamycin was employed as template. The 185-bp-length PCR product was separated on a 2% agarose gel, excised from the gel and sequenced as described above for verification.

Treatment of plants and leaf discs: Exposure to SO₂ was carried out in a 40 liter growth chamber under control of SO₂-Control System (WGA-50-MAS, Emproco, Ashkelon, Israel), continuously supplied with calibration gas cylinder containing 250 ppm SO₂ in air (Scientific & Technical Gases, Newcastle, UK), designed to maintain stable SO₂ concentration. Tomato and Arabidopsis WT, SO RNAi and overexpression lines were exposed to 1 and 2 ppm SO₂ for 2 or 4 hours, under light (40μ einsteins m⁻² s⁻¹) at 25° C. with a relative humidity of 85-95%, and sampled immediately or after 24 for expression analysis. For chlorophyll content and leaf damage analysis, plants were left to recover for 4 days in the growth room and then analyzed. Plants at identical conditions without exposure to SO₂ served as control.

For measuring the effect of SO₂ fumigation on whole fruits, fruits were exposed to SO₂, for 4 hours at 20 ppm, then for 24 hours at 2 ppm. The size and character of the fungal lesion was assessed 72 hours following exposure. For measuring the effect of SO₂ fumigation on calyx and peduncle structures, tomato berries with stalk and leaves were exposed to 2 ppm SO₂, for 6 hours. Assessment of SO₂ damage was performed after 40 hours recovery. Damage was scored according to the following scale: 5=fully recovered, 4=slightly damaged (beginning of petal curling), 3=medium damage (50% petal curling), 2=damage (75% appear curled and wilted), 1=severe damage (petals and calyx sepals appear wilted). n=7 to 13 fruits per group.

Anthracnose infection: Fruit of LeSO over expression and LeSO null RNAi lines were inoculated with Colletotrichum coccodes hyphal mats. This represents a massive infection titer of anthracnose. The fungal mat was prepared in the following manner. Fungi were grown in 40 mL liquid Mathur's medium at pH 5.0, inoculated to a density of 1×10⁶ spores/flask. [Mathur's medium contains the following reagents (per liter): 2.5 g MgSO₄*7H₂O, 2.7 g KH₂PO₄, 1.5 g Bacto peptone, 1.5 g Bacto yeast extract (Difco, Detroit, Mich.), 15 g sucrose, and 250 mg chloramphenicol (primary medium)]. Cultures were incubated in a 24° C. incubator with shaking at 150 rpm for 3 days, and were harvested by filtration through sterile Büchner funnel-fitted filter paper. The hyphal mat was washed twice by filtering with 40 mL of sterile distilled water and discs were applied to the fruit. The fruit was prepared in the following way: 0.8 cm diameter circles of epidermis were peeled and 0.8 cm of a hyphal mat was applied. Infection was evaluated according to the diameter and color of the fungal lesion (in mm).

Exposure of leaf discs to NaHSO3: For leaf disc treatment, 3 to 4-week-old WT and transgenic Arabidopsis and 5 weeks old WT and transgenic tomato plants, 7 and 9 mm in diameter, respectively were placed in 90 mm diameter plates on a filter paper moistened with 2 ml of 50% MS salt solution with or without 7 mM Na₂SO₃ for 24 hours under constant illumination (100μ einsteins m⁻² s⁻¹) and then were photographed and analyzed for chlorophyll content.

Determination of sulfate, chlorophyll, leaf damage level and relative leaf area: For sulfate determination, leaves of Arabidopsis and tomato WT and SO modified plants were sampled immediately after exposure to 2 ppm SO₂, extracted in double distilled water (1:3, w/v), heated for 5 min at 95° C. and then determined by ion exchange chromatography system (DX 600; Dionex) using IonPac® column (AS 4A-SC; Dionex) for separation and an electrochemical conductivity detector (ED 50; Dionex) combined with an upstream-inserted micromembrane suppressor (ASRS-Ultra II 4 mm; Dionex). The retention time of 3.57 and 3.90 distinguished sulfite from sulfate, respectively and plant sulfite levels were below detection limits (<1 ppm). For statistical analysis, each treatment was compared to its own control using two-tailed t-test. Total chlorophyll content was measured in extracts of the fully expanded leaves as described before (Graan and Ort, 1984). Severity scale for leaf damage was designated as follows: 1, no damage; 2, less than 30%; 3, 30% to 50%; and 4, more than 50% of the leave area was damaged. The severity scale average was then multiplied by the total number of damaged leaves to determine the damage level. Relative leaf area was calculated as the ratio of sum of leaf length multiplied by leaf width divided by the leaf area of untreated plants. Mean±s.e.m of each treatment was presented.

Protein extraction, fractionation, Western blot and in gel SO activity: For protein extraction, leaves of tomato and Arabidopsis samples were ground using a pestle and mortar in extraction buffer (4 mL g⁻¹ fresh weight) containing 0.25 M Sucrose, 50 mM Tris-HCl (pH 8.5), 3 mM EDTA, 1 mM sodium molybdate and a cocktail of protease inhibitors including aprotinin (10 μg ml⁻¹), leupeptin (10 μg ml⁻¹), and pepstatin (10 μg ml⁻¹). The homogenate was centrifuged at 4,000 g for 5 min. The resulting supernatant was subjected to centrifugation at 18,000 g for 20 min and the pellet was tenderly dissolved in the above extraction buffer supplemented with Triton X-100 in a final concentration of 0.025%. For direct measurement of SO protein, proteins were separated by SDS-PAGE carried out in 10% polyacrylamide gels, and blotted onto polyvinylidene difluoride membranes (Immun-Blot Membranes, Bio-Rad). Blotted proteins were subjected to immunodetection with antibodies raised against tomato and Arabidopsis SO synthetic polypeptide, RHPSLKINAKEPFNAE (SEQ ID NO: 27). Primary antibodies were diluted 500-fold in TBS, and secondary antibodies (anti-guinea pig IgG, Sigma) were diluted 1000-fold in TBS. Phosphatase activity was visualized by staining with 5-bromo-4-chloro-3-indolyl phosphate and NBT. In-gel assay of SO activity, following H₂O₂ production, was examined after protein refractionation with Native-PAGE. A modified chromogenic horseradish peroxidase (HRP) assay was employed in which H₂O₂ serves as a proton-accepting substrate while o-dianisidine serves as a proton donor (Manchenko, 1994; Yesbergenova et al., 2005). The modified reaction mixture contained 2.5 mM o-dianisidine, 4.5 U mL⁻¹ HRP and 0.4 mM sodium sulfite. The reaction was stopped by immersion of the gels in doubly distilled water. For verification the detected activity bands were excised and subsequently refractionated by denaturing SDS-PAGE and immunobloted with SO-specific antisera. The bands detected after western blot and in-gel assay were scanned in an Arcus 1200 Scanner (Agfa, Mortsel Belgium) and quantified by NIH Image Software (Version 1.6).

Determination of protein concentrations: Concentrations of total soluble protein were determined according to Bradford (1976).

Kinetic assays of SO and ROS-generating activity: H₂O₂-generating activities in leave extracts of WT and SO modified plants was detected in reaction mixture containing 10 μg soluble protein, 0.85 mM 4-aminoantipyrine, 3.4 mM 3,5 dichloro-2-hydroxobenzene sulphonate 4.5 U mL⁻¹ HRP in 1 mL of 50 mM phosphate buffer (pH 7.5). The colorimetric assay is based on 3,5 dichloro-2-hydroxobenzene sulphonate, which couples oxidatively to 4-aminoantipyrine in the presence of H₂O₂ and HRP to yield a red quinonemine dye (Fossati et al., 1980, Yesbergenova et al., 2005). The H₂O₂-generating activity was assayed spectrophotometrically at 515 nm after the addition of 0.4 mM sodium sulfite. SO activity following the reduction of ferricyanide at 420 nm, in reaction mixture containing 10 μg soluble protein, 0.395 mM ferricyanide, 0.4 mM sodium sulfite in 1 mL of 20 mM Tris-HCl buffer (pH 8). One unit of SO activity was defined as the conversion of 1 μmol of sulfite min⁻¹ (Eilers et al., 2001). For both assays, a reaction mixture without sodium sulfite served as control blank.

Quantitative Real Time RT-PCR: For each RT reaction, 0.5 μg of Arabidopsis total RNA was reverse-transcribed with an iScript™ cDNA Synthesis Kit using modified MMLV-derived reverse transcriptase (Bio-Rad, Hercules, Calif.) and a blend of oligo-d(T) and random hexamer primers according to the manufacturer's instructions. Quantitative RT-PCR reactions contained 1:13 (v/v) of the first-strand cDNA as the template, specific primers and iQ™ SYBR® Green Master Super Mix (Bio-Rad, Hercules, Calif.) in a final volume of 15 μl, using the primers listed in Table 1 below. Amplification with Arabidopsis materials was performed for 40 cycles, consisting of initial pre-heating of 95° C. for 3 min, 20 sec at 95° C., 20 sec at 65° C. and 30 sec at 72° C. Fluorescence increments of each reaction were simultaneously monitored with the iCycler iQ™ Multicolor Real-Time PCR detection system (Bio-Rad, Hercules, Calif.). The PCR products were separated on a 2% agarose gel, excised from the gel and sequenced for verification. Reactions normalized with ACTIN 2 (At3g18780) and Elongation factor 1-alpha (At5g60390) for Arabidopsis, or ACTIN Tom4 (U604480) and Elongation factor 1-alpha (SGN-U196120) as housekeeping genes revealed similar results. Results normalized with ACTIN2 or ACTIN Tom41 are presented, in units of relative expression.

TABLE 1
Quantitative real-time PCR Primers

| | | |
|---|---|---|
| Transcript | Primer | PCR product |

Arabidopsis thaliana primers

| | | |
|---|---|---|
| WRKY6 (senescence-related | forward AAGGATTTCGTGTGAAGAAGGAAGAAC | 155 |
| transcription factor; | (SEQ ID NO: 28) | |
| At1g62300), | reverse CAACTCATTITTCGCACGCTTATCT | |
| | (SEQ ID NO: 29) | |
| ERD1/SAG15 | forward TCTCAAGAGATTGGGAGCAAATATGAA | 190 |
| (senescence-associated gene | (SEQ ID NO: 30) | |
| 15; At5g51070) | reverse GAACTGTTCCAGTACATTTTTCGCTTTT | |
| | (SEQ ID NO: 31) | |
| ACX1 (acyl-CoA oxidase 1; | forward GTGTTCAAAAGGCTGAGGATTGGTTA | 134 |
| At4g16760), | (SEQ ID NO: 32) | |
| | reverse CTCTTGGAATCCTTGTTCCTGATTCTC | |
| | (SEQ ID NO: 33) | |
| ER5/LEA (ethylene- | Forward TTACATCCTCAAGAGTGCCACAAGGAC | 191 |
| responsive 5/late | (SEQ ID NO: 34) | |
| embryogenesis-like protein; | reverse CGAAGGTTAGCCCAATGTCTAGTTGATA | |
| At2g46140) | (SEQ ID NO: 35) | |
| XERO1/TAS14 | forward AGACTCACCAACAGCTTGACCAATTT | 165 |
| (dehydrin; At3g50980) | (SEQ ID NO: 36) | |
| | reverse CACCTAGTCCATCATCCGAGCTAGAG | |
| | (SEQ ID NO: 37) | |
| SRG1 | forward AAGAGTGGGGATTTTTCCAGCTTGT | 191 |
| (senescenc-related gene 1; | (SEQ ID NO: 38) | |
| At1g17020) | reverse TGCCCAATCTAGTTTCTGATCTTCTGA | |
| | (SEQ ID NO: 39) | |
| SO (Sulfite oxidase; | Forward AAAGACATCAGGTCCCTCCCAAAGTA | 130 |
| At3g01910) | (SEQ ID NO: 40) and | |
| | reverse CAATAGCAGAAACATCCCATCCAAC | |
| | (SEQ ID NO: 41) | |
| SiR (Sulfite reductase; | Forward CAATGTTTGAAAAGGTTGGTCTGGACT | 145 |
| At5g04590) | (SEQ ID NO: 42) | |
| | reverse CCTCCTAGCCAAACCTGATAGCTGTT | |
| | (SEQ ID NO: 43) | |
| SQD1 (UDP-sulfoquinovose | forward TATGGTAAAGGTGGTCAGACGAGAGG | 200 |
| synthase; At4g33030) | (SEQ ID NO: 44) | |
| | reverse GTCATCTTTTTCACGTCTAGCCCAAG | |
| | (SEQ ID NO: 45) | |
| MST1 (mercaptopyruvate | forward TGATCAGGTCAAGAACAATATGGAGGA | 161 |
| sulfurtransferase; At1g79230) | (SEQ ID NO: 46) | |
| | reverse AAGAATCAAACATCTGAGGAAAAGGGATA | |
| | (SEQ ID NO: 47) | |
| MST2 (mercaptopyruvate | forward GCTCGTGTATGGTGGATGTTTAGAGTC | 193 |
| sulfurtransferase; thiosulfate | (SEQ ID NO: 48) | |
| sulfurtransferase like protein; | Reverse AAGTTATTGGGCTAATTGTTTGTCCTTGA | |
| At1g16460) | (SEQ ID NO: 49) | |
| ACTIN 2 (At3g18780) | forward TTGTGCTGGATTCTGGTGATGG | 167 |
| | (SEQ ID NO: 50) | |
| | reverse CCGCTCTGCTGTTGTGGTG | |
| | (SEQ ID NO: 51) | |
| Ef1-α (Elongation factor | forward CAGGACATCGTGATTTCATCAAGAAC | 190 |
| 1-alpha; At5g60390) | (SEQ ID NO: 52) | |
| | reverse TCCATCTTGTTACAACAGCAAATCATCT | |
| | (SEQ ID NO: 53) | |

Tomato primers

| | | |
|---|---|---|
| Actin Tom41 (U60480) | forward CATGCCATTCTCCGTCTTGA | 71 |
| | (SEQ ID NO: 54) | |
| | reverse CGCTCGGTCAGGATCTTCAT | |
| | (SEQ ID NO: 55) | |
| Ef1-α (Elongation factor | forward CCTACTTGAGGCTCTTGACCAGATT | 160 |
| 1-alpha (SGN-U196120) | (SEQ ID NO: 56) | |
| | reverse AAAAGTGACAACCATACCAGGCTTAAT | |
| | (SEQ ID NO: 57) | |
| SO (Sulfite oxidase; GenBank | forward CCTGGAGGATGTGAGTGTTGTAAAG | 145 |
| accession No. DQ853413) | (SEQ ID NO: 58) | |
| | reverse AGTTCTCTGGTATCTGGTGGCTTC | |
| | (SEQ ID NO: 59) | |
| SiR (Sulfite reductase; | forward AAGTTGTGAAAGCTCGGAATGATAACT | 185 |
| SGN-U214723) | (SEQ ID NO: 60) | |
| | reverse TTCTCCATCCTCATCAGATACAACAAC | |
| | (SEQ ID NO: 61) | |
| MST1 (Thiosulfate | forward TCAAGAGTATCAGGTTGCACATATTCC | 183 |
| sulfurtransferase; SGN- | (SEQ ID NO: 62) | |
| U320318) | reverse CTAAAGATTCCCTTCCCATCATAGACA | |
| | (SEQ ID NO: 63) | |
| MST2 (Thiosulfate | forward TTGCACCTATTACCTTTCTGACCAAAT | 178 |
| sulfurtransferase; SGN- | (SEQ ID NO: 64) | |
| U320318) | reverse CTCCCAGGTACATGACCACTTCTTATT | |
| | (SEQ ID NO: 65) | |
| SQD1 (sulfolipid biosynthesis | Forward GTTGACAACCTTATCCGTCGATTATTT | 195 |
| protein; SGN-U217001) | (SEQ ID NO: 66) | |
| | Reverse GACTACAGCATCAGGTTCAAAGGATTT | |
| | (SEQ ID NO: 67) | |

Sequence analysis: Sequence analysis was performed with the ABI Prism Big Dye Terminator Cycle Sequencing Ready Reaction Kit on an ABI Prism 310 cycle sequencer (PE Applied Biosystems, Warrington, UK).

Accession number: The GenBank Accession number of tomato Sulfite Oxidase is DQ853413.

### Experimental Results

### Example 1

### Expression of SO in Plants

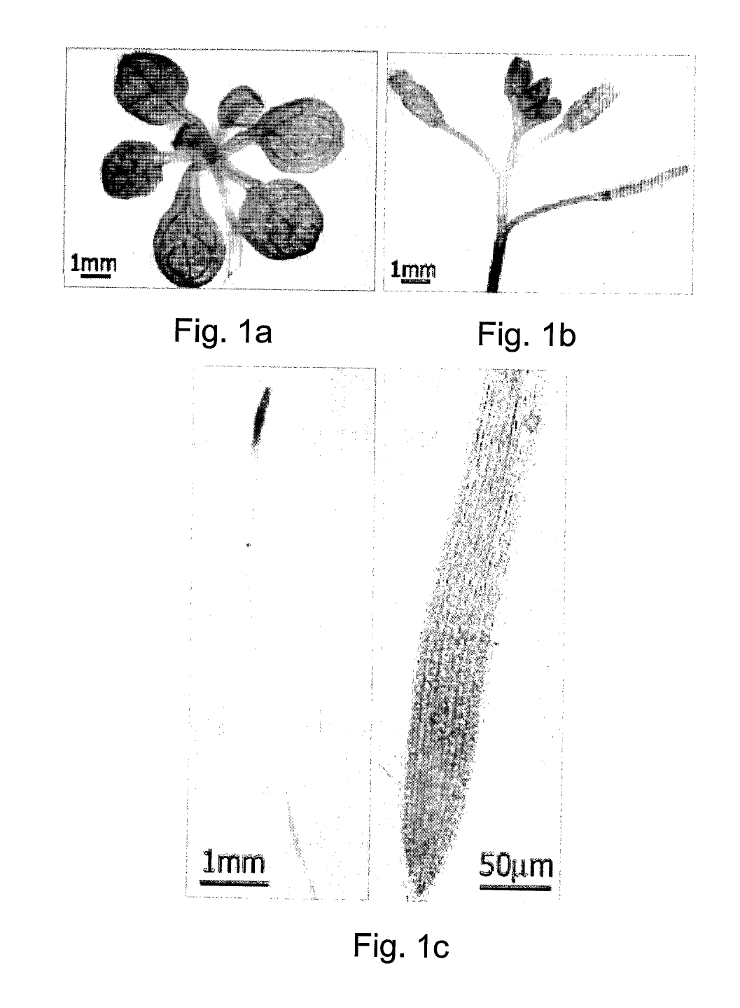

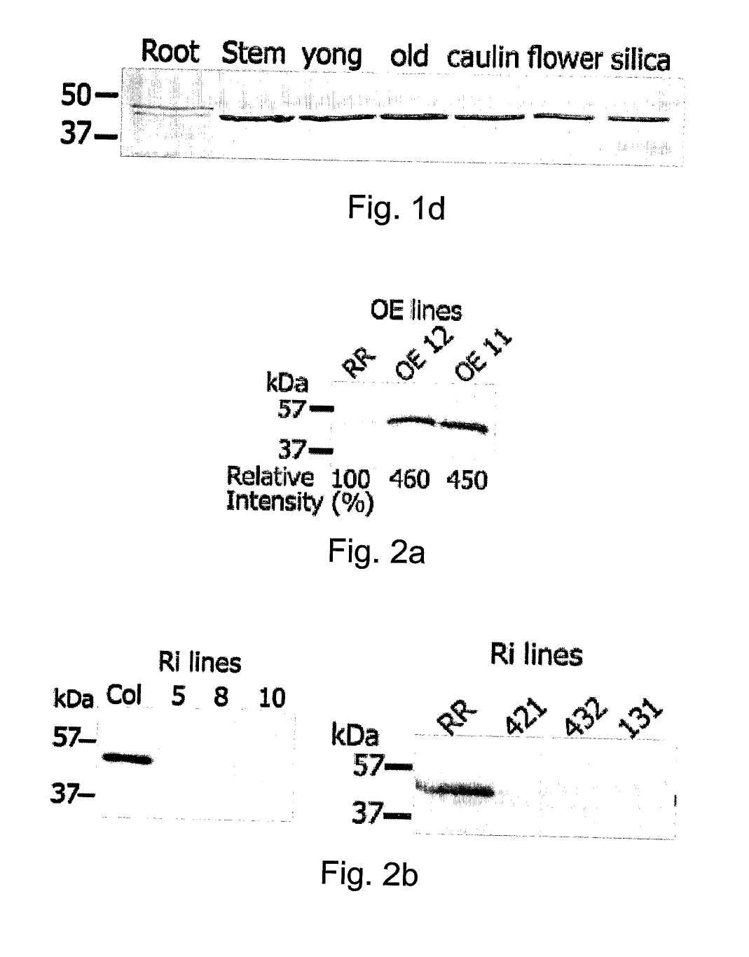

SO is present as a single gene in Arabidopsis. Digital northern and response activities of a 2,204-slide size microarray collection by GENEVESTIGATOR (https://www.genevestigator.ethz.ch) (Zimmermann et al., 2004) showed that SO is constitutively expressed in all plant organs and is was not significantly induced in any of the 75 diverse experimental conditions represented in the microarray (results not shown). To obtain a more accurate image of SO expression in plants and plant tissues, a 1,562 bp promoter fragment (90500-92062 bp in the BAC F1C9 sequence) (SEQ ID NO: 68) was used to direct the expression of the β-glucuronidase (GUS) reporter gene in Arabidopsis. Out of 20 transformed lines 5 had significant levels of expression. Histochemical analysis showed GUS expression in all tissues (see FIG. 1a-1c) with a higher degree of staining in stem, hypocotyls and root vasculatures (FIG. 1a) and at the root tip and young inflorescences (FIGS. 1b and 1c).

Immuno-detection confirmed constitutive expression of a 45 kD polypeptide in all plant organs (FIG. 1d) except root tissue, in which SO migrated in 2 distinct forms. Without wishing to be limited by a single hypothesis, it is proposed that the presence of isoforms may represent the products of different post-translational modifications. The closely correlated results of SO microarray analysis, promoter expression (SO::GUS expression, FIGS. 1a-1c) and protein accumulation (FIG. 1d) indicate that there is direct correlation between SO gene expression and levels of immunodetectable SO protein in plants.

### Example 2

### SO Modified Plants

In order to evaluate the role of SO, its expression levels were modulated in Arabidopsis (AtSO) and tomato (LeSO) plants.

Cloning and sequencing of Tomato SO: Full length tomato SO (LeSO, GenBank Accession number DQ853413) was cloned and sequenced using SO specific primers, as described in Experimental procedures hereinabove. The resultant full length tomato SO (LeSO) exhibited 77% identity with Arabidopsis AtSO (Eilers et al., 2001). The LeSO sequence includes the C-terminus tripeptide ANL, which corresponds to the consensus peroxisomal targeting signal type 1 (A/C/G/S/T-H/K/L/N/R-I/L/M/Y) (Mullen et al., 1997a; Mullen et aL, 1997b). While not wishing to be limited to a single hypothesis, it seems likely that the presence of the peroxisomal targeting signal in both the tomato and Arabidopsis genes indicates functional similarity between SO in the two plant species.

Modified expression of SO in transgenic plants: In order to determine the effect of modulated SO activity on phenotypic character in plants, transgenic plants including both SO-null AtSO and LeSO RNA interference (RNAi) lines, lacking immuno-detectable SO polypeptide (13 and 3 independent lines in Arabidopsis and tomato, respectively) and constitutive over-expression (OE) independent lines exhibiting up to 4-fold higher protein levels (3 independent lines in tomato) were generated using Agrobacterium-mediated transformation, as detailed hereinabove. When proteins from the SO-modified transgenic plants were separated, blotted and immunodetected with antibodies raised against a tomato and Arabidopsis SO synthetic polypeptide, up-regulation of SO expression in all the overexpressing lines (FIG. 2a) and down-regulation of SO expression (absence of immunoreactive SO) in all the SO-null [FIG. 2b, (Arabidopsis) and (tomato)] lines assayed was clearly shown: SO-null lines lacked immunodetectable SO protein (FIG. 2b), while the SO-overexpressing lines exhibited up to 4-fold higher SO protein levels (FIG. 2a).

Extracts of wild type, RNAi and OE lines were examined for SO activity by employing an assay that measures the reduction of ferricyanide. The results showed that the activity in Arabidopsis and tomato RNAi and OE extracts was either more than 68% lower or more than 231% higher, respectively, of that shown in wild type plants (see FIGS. 3a-3b).

To further examine SO-dependent activity, an independent assay was developed based on the ability of SO to generate H₂O₂ during sulfate formation. In this case, the measured H₂O₂ product was 56 or 73% higher in wild-type than extracts of SO-null Arabidopsis and tomato respectively, and OE modified tomato plants were 3.3-fold higher than in Arabidopsis and tomato wild-types, respectively (FIGS. 3a and 3c, right insert). If the residual activity measured in SO-null lines is considered as non-SO dependent activity, activities in OE lines were 6.4-fold higher than the wild-types in the transgenic SO OE tomato, consistent with the range of protein fold increase detected by Western blot analysis.

In an attempt to directly correlate the H₂O₂ generation with SO, we modified a chromogenic horseradish peroxidase (HRP) in-gel assay in which accumulating H₂O₂ serves as a proton-accepting substrate while o-dianisidine serves as a proton donor. Extracts were fractionated under native PAGE conditions, and broad orange coloured bands of H₂O₂ generating activity could be detected in tomato (FIG. 3b) plants. SO activity was less intense in wild types Col and RR, and was absent in Ri lines, but was significantly greater in intensity in transgenic tomato OE leaves. The area of the activity bands was excised and refractionated by denaturating SDS-PAGE, and immunobloted with SO-specific antisera (FIGS. 3b and 3d, right insert). Polypeptides cross-reacting with SO antisera of correct molecular weight were detected in bands of enzyme activity from OE and wild-type plants, but were absent from SO-null RNAi lines. The intensity of immunoreactivity was 5-fold higher in the SO-OE tomato plants than in tomato wild-types, consistent with the activity gel measurements.

Thus, sulfite oxidase catalytic activity, measured in a variety of assays, correlates with the expression levels in the transgenic plants, being highest in the transgenic overexpressing lines (FIGS. 3a-3d). Thus, using genetic engineering techniques such as RNAi or overexpression, transgenic plants having modified SO expression can be generated.

Sulfite metabolism in SO-modified transgenic plants: The role of SO, and of sulfite oxidation in sulfite metabolism in plants has not yet been determined. In order to examine the effect of modulation of SO expression on plant metabolism, and on the susceptibility of transgenic SO-modified plants to sulfite-related toxicity, total sulfate levels were monitored in leaf extracts after treatment of plants with 2 ppm SO₂ for 2 hours (FIG. 3). Sulfate was chosen since sulfite is difficult to measure as it is maintained at low levels in plant tissues, difficult to recover and rapidly oxidizes in extracts. Leaves of representative Arabidopsis and tomato plants were treated with 2 ppm SO₂ for 2 or 4 hours, respectively.

Increases of 24.7 and 43.5% in sulfate accumulation for wild type and all transgenic SO OE tomato lines, respectively, were detected. In contrast, the effect of SO₂ treatment on sulfate levels in SO-null RNAi plants resulted in smaller increases of 2.7 and 6.7% in Arabidopsis and tomato, respectively (FIGS. 3e and 3f). Thus, accumulation of sulfate in plant tissue in response to SO₂ application is directly correlated with the SO levels of the plants.

### Example 3

### Transgenic Plants Over- or Under Expressing SO Exhibit Modified Susceptibility to Sulfite and Sulfite-Producing Substances

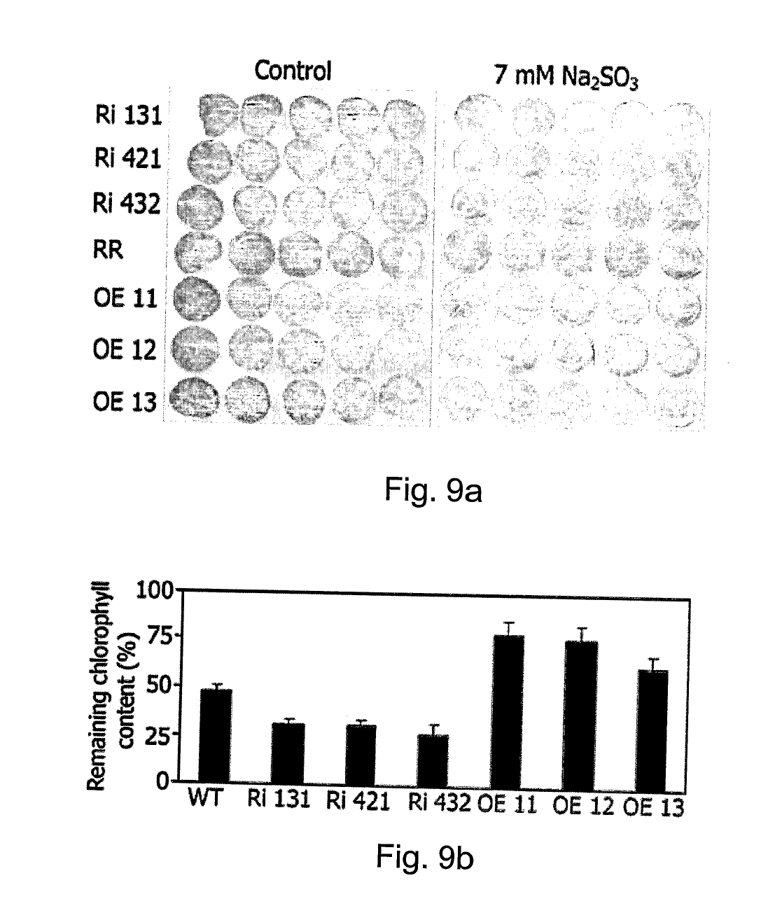

Susceptibility to Na₂SO₃: To further examine the response of SO modified plants to sulfite and sulfite-producing compounds, leaf discs of representative wild type and SO-modified transgenic plants were treated with 7 mM Na₂SO₃, and then viability and health of the tissue determined. Leaf discs of all SO-null RNAi Arabidopsis and tomato lines showed significantly higher chlorosis and damage than wild type and over expression lines (Arabidopsis, FIG. 4a; and tomato, FIG. 9a). Further, since chlorophyll content is a sensitive indicator of leaf health, the chlorophyll content of the wild type and transgenic plants was determined. After 24 hours exposure, a reduction in the level of chlorophyll of 30% to 50% was detected in all Arabidopsis SO-null RNAi lines compared to a reduction of 10% in wild type lines (FIG. 4b). Chlorophyll content of all tomato SO-null RNAi lines exhibited similar enhanced sensitivity to Na₂SO₃ (FIG. 9b), while that of all transgenic tomato plants overexpressing LeSO was significantly less affected (FIG. 9b).

These results show that susceptibility to sulfite-related damage in transgenic plants having modified SO expression, such as SO-null RNAi and overexpressing OE lines, closely correlates with the SO levels, with SO overexpression conferring enhanced resistance, and reduced SO expression conferring enhanced susceptibility to sulfite-related damage.

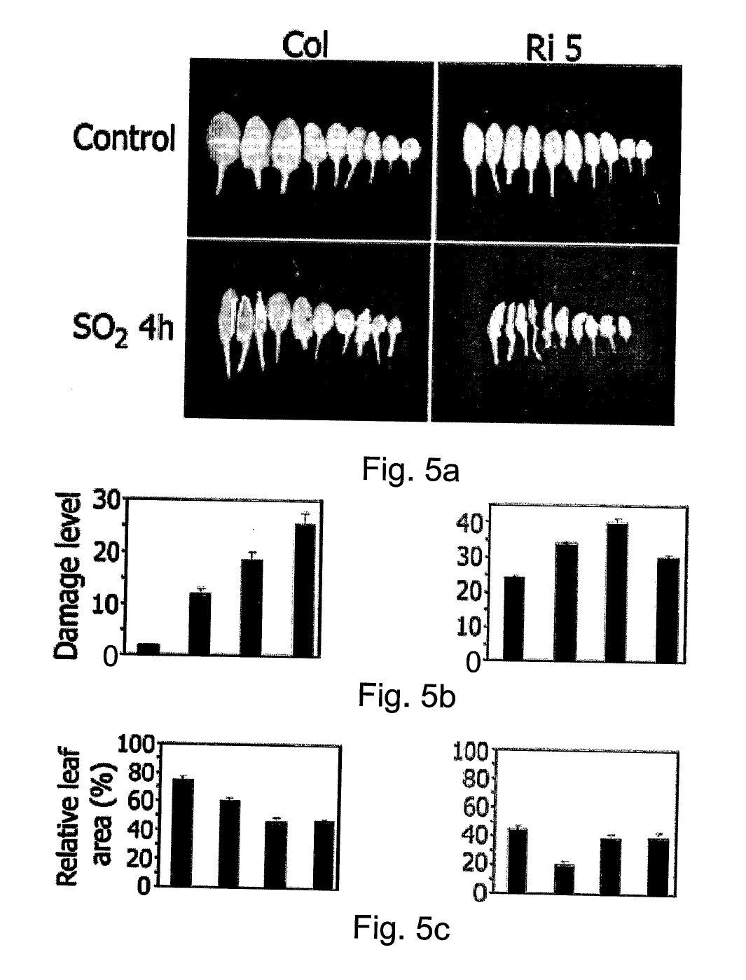

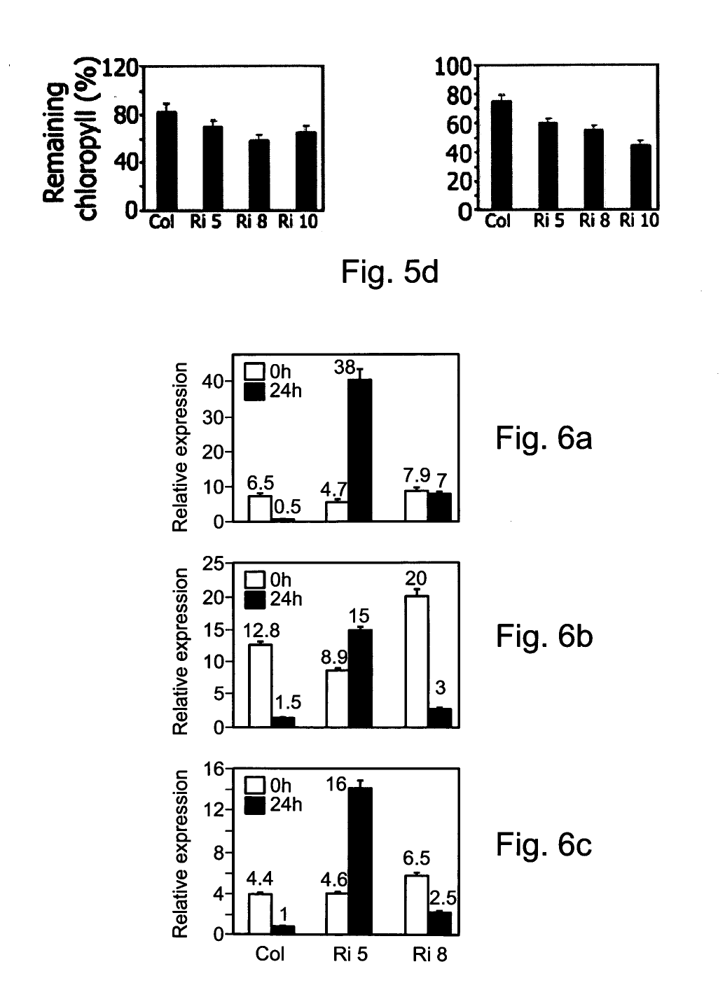

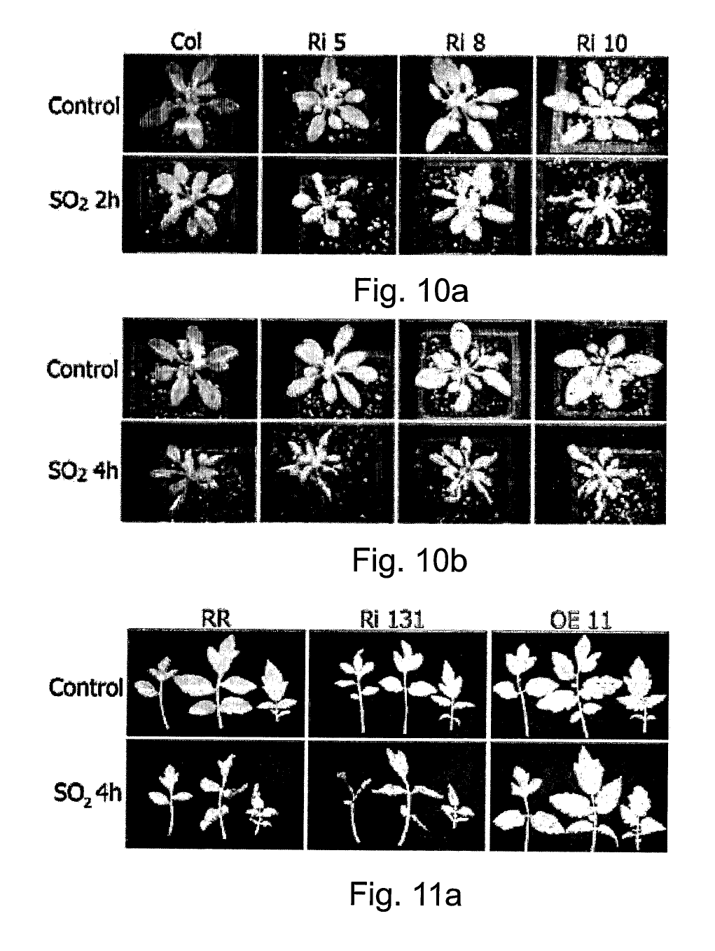

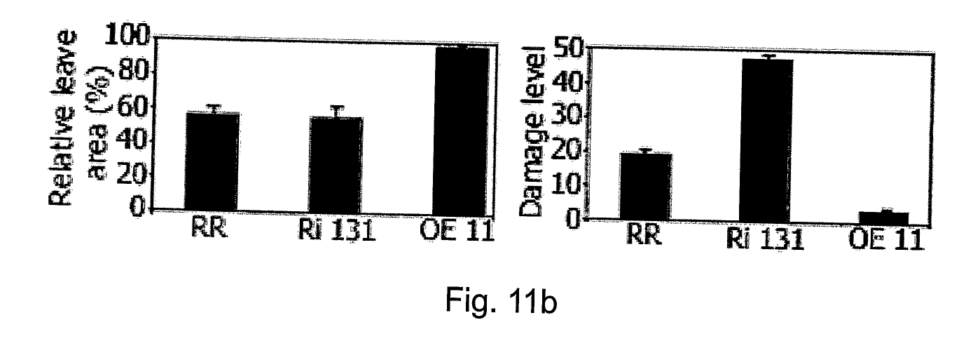

Sulfur dioxide toxicity in wild type and transgenic SO-modified plants: SO₂ is a highly cell-permeable toxic gas which can reach levels of 2 ppm in heavily polluted regions, and is widely used for fumigation of flowers and fruits. When Arabidopsis wild-type and transgenic lines were exposed to 1 ppm of SO₂ for 2 or 4 hours no significant damage was observed in any of the leaves within 4 days (data not shown). However, when wild type Arabidopsis (FIGS. 5a-5c and FIG. 10a) and tomato (FIG. 11a) plants were exposed to 2 ppm SO₂ for 2 or 4 hours, and examined 4 days later, increasing levels of leaf damage were consistently found, correlating with the length of exposure. Quantitative analysis of the leaves of all of the SO-null RNAi lines showed damage index values that were 5-10 fold the damage levels in wild type plants after 2 hours of exposure to 2 ppm (FIG. 5b). After 4 hours of exposure leaf growth was severely arrested in all of the wild type and SO-null RNAi lines (50% of non-treated controls) (FIG. 5c). The index of residual chlorophyll showed similar sensitivity of the SO-null plants to SO₂ damage (FIG. 5d). Importantly, leaves from all of the SO-null (RNAi) tomato plants sustained greater damage than those from wild type (RR) or any of the SO overexpressing OE plants when exposed to 2 ppm SO₂ for 4 hours (FIG. 11). Although the calculated relative leaf area of all of the SO-null plants was similar to wild type (RR), the SO-null leaves were significantly more damaged, while leaves of all the SO overexpressing OE plants showed minimal symptoms of SO₂ toxicity (FIG. 11b).

These results indicate the possibility that a distinct threshold for SO₂ toxicity exists in some wild type plants, and indicate, as shown by the modulation of SO levels, that the physiological capacity of the plant to detoxify SO₂ by SO activity is a critical point for SO₂ tolerance, and that the tolerance is enhanced by increasing SO activity.

Effect of exposure to SO₂ on senescence and wounding-associated gene expression in transgenic SO-modified plants: Plants exposed to SO₂ poisoning react in a manner similar to that of leaf senescence and wounding stress. In order to further determine the effect of modulation of SO expression on sulfite-related toxicity in plants, the activity of genes that are known to be associated with leaf senescence and wounding processes was monitored in wild type and SO-modified transgenic plants.

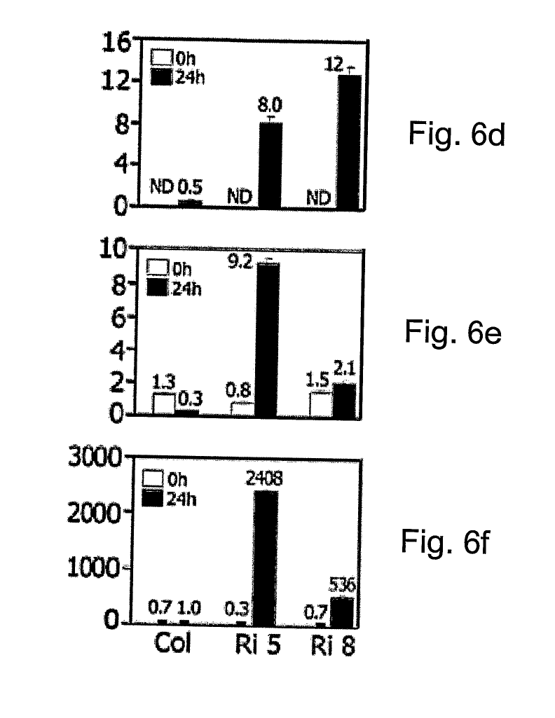

WRKY6 (senescence-related transcription factor), ERD/SAG15 (senescence-associated gene) and ACX1 (acyl-CoA oxidase 1) are triggered during early senescence and plant defense responses. Treatment of wild type and SO-modified transgenic plants with 2 ppm SO₂ showed rapid 4-10 fold accumulation of these transcripts in all lines (FIGS. 6a-6c). However, the induction level remained elevated 24 hours later in Arabidopsis SO-null AtSO RNAi lines but not in wild type plants. A different set of marker genes that emphasize late processes in senescence and stress were also monitored. These include; ER5/LEA (ethylene-responsive 5/late embryogenesis-like protein), XERO1/TAS14 (dehydrin) and SRG1 (senescence-related gene 1). These genes were shown to be activated in later stages of leaf senescence, drought and wounding. Regarding the expression of these genes, wild type plants showed little change, however, SO-null RNAi lines displayed high levels of induction after 24 hours (FIGS. 6d-6f). Taken together, as the levels of these transcripts reflect cellular stress responses, the results indicate that SO levels play an important role in preventing stress induced by toxic levels of SO₂.

Effect of Exposure to SO₂ on Sulfite Utilizing Enzymes in Transgenic SO-Modified Plants:

SO, SiR, SQD1, and MST are all plant enzymes that use sulfite as substrate and could play pivotal roles in SO₂ metabolism. Thus, the expression level of SO, SiR, SQD1 and MST were simultaneously monitored in wild type and transgenic SO-modified plants.

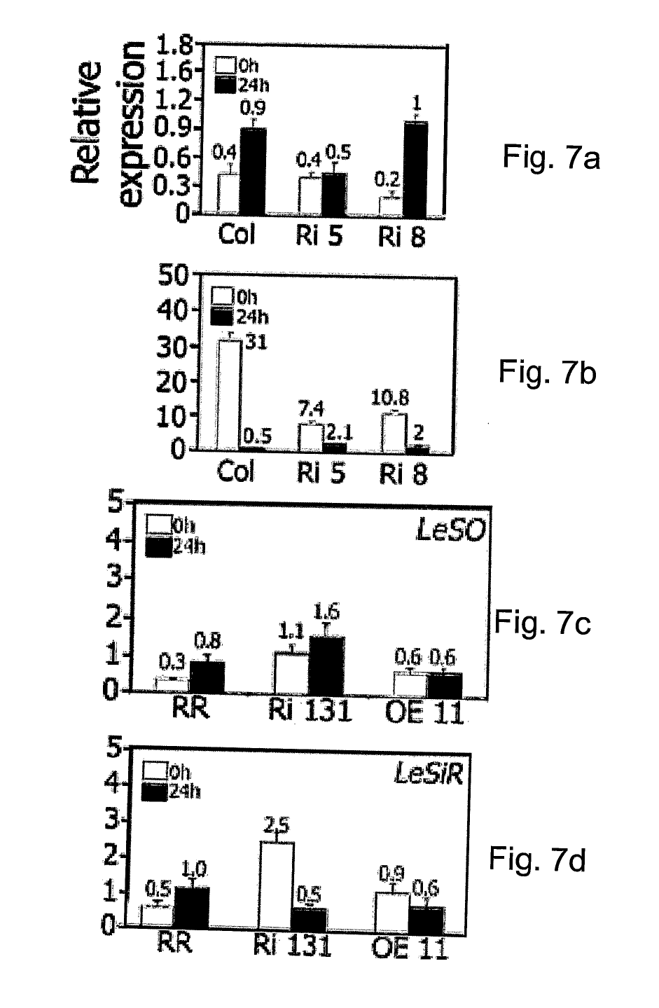

When Arabidopsis plants were exposed to 2 ppm SO₂ for 2 hours and examined immediately, the levels of SO were reduced by at least 2 fold or more in wild-type lines. After 24 hours the levels returned to normal (FIG. 7a). Similar results were obtained for wild type LeSO (FIG. 7b). Similarly, immuno-detection of SO revealed only moderate (20%) increase in the amount of SO polypeptide in wild type tomato and Arabidopsis plants (data not shown). Thus, Arabidopsis and tomato SO transcript and protein levels are not highly sensitive to application of SO₂. The relative levels of AtSO and LeSO transcripts in SO-null RNAi plants changed to a greater extent but their absolute levels are inherently very low (approximately, 10⁴ and 10⁸ lower than WT and OE transcripts, respectively), and any change in those levels may be a reflection of changes in general RNAi-specific processes.

In contrast, AtSiR expression was enhanced more than 30 fold immediately after treatment in wild type Arabidopsis plants and between 7 and 10 fold in RNAi plants (note different scales; FIG. 7b). When measured 24 h later, AtSiR expression was elevated in RNAi lines by 2 fold but not in wild type, where it returned to lower than normal levels (FIG. 7b).

Tomato plants showed a different response of the sulfite-utilizing enzymes to SO₂ exposure. The LeSiR transcript was not induced in response to SO₂ treatment in wild type RR plants (FIG. 7d), although a significant increase was obtained in RNAi plants but not in OE plants (FIG. 7d). These results indicate that the level of transcripts of AtSiR and to lesser extent LeSiR are responsive to SO₂ levels, particularly under conditions of limitation in SO activity.

SQD1, MST1 and MST2 represent genes that catalyze the diversion of sulfite to other assimilatory pathways. In order to determine whether their expression was also regulated by fumigation with SO₂, levels of transcription for these genes was assessed.

As shown in FIGS. 8a-8f, immediately after exposure to (0 h) to SO₂ no significant differences in transcript levels were detected. However, in both Arabidopsis (FIGS. 8a-8c) and tomato (FIGS. 8d-8f), after 24 hours, SO-null (RNAi) plants, but not WT or transgenic SO overexpressing (OE) plants contained elevated levels of transcripts after exposure to SO₂/sulfite treatment. These results indicate that the late responsive (24 hour) SO-dependent transcripts up-regulation of SQD1, MST1 and MST2 is distinct from the early responsive (t=0 hours) SiR.

The modulation of sulfite levels by SO expression uncovers the physiological cross-relationship between the oxidative and reductive pathways in sulfite metabolism in which AtSO may serve as a physiological safety valve. Without wishing to be limited by a single hypothesis, it is possible that, although the a cellular increase in AtSiR expression level may be able to alleviate some toxic effects of initial SO₂ exposure, SO₂ exposure above these levels requires further detoxification that only AtSO can provide. Regulation of AtSiR may be connected to SO activity. In this respect it is of interest that peroxisomes are closely associated with chloroplasts facilitating potential metabolite cross-talk.

SO Modulation in Fruit and Related Structures makes it Amenable to Protection by Sulfur Dioxide Fumigation

SO₂ gas is toxic to microbes at low concentrations, and in solution, its toxicity is a function of pH.

For tomato, commercial post-harvest preservation relies on costly storage of unblemished berries at reduced temperature (12° C.). Any pre-existing injury or presence of fungal inocula is likely to result in decay and spread of the decay between adjacent berries. However, tomato berries and especially the peduncle (fruit stalk that holds a fruit bunch) and calyx (leaf-like sepals at the fruit top) are also susceptible to SO₂ treatment. The integrity of these structures is important as many fresh marketing procedures leave these structures intact to give tomato fruit a ‘freshly picked’ appearance. Adapting SO₂ technologies for tomato storage should significantly reduce storage costs and loss of spoiled berries.

Many practical permutations exist for SO₂ application. For example, SO₂ is applied at a relatively high concentration soon after packaging and then maintained at much lower concentartions. High concentrations kills the relatively SO₂-resistant conidia of pathogens present on the berry before they can penetrate the surface. Lower SO₂ concentrations kills aerial mycelia, which are more sensitive to SO₂ than conidia. In this way, berry-to-berry spread of the pathogen is inhibited.

In order to test the usefulness of enhanced SO₂ expression in tomato fruit, the fungus Colletotrichum coccodes, which causes Anthracnose (ripe rot), the most important and most common fruit rot in tomatoes, was chosen to test the susceptibility of transgenic and wild type tomato fruit to mold growth in the presence of SO₂. Anthracnose infections also allow entry of soft rot organisms that further decay the fruit during transport and storage.

To show that SO₂ can inhibit fungal inections such as anthracnose, approximately 20 fruit of different stages of ripening were collected and divided into red (mature), late breaker and early breaker phases. Fruit of LeSO over expression and LeSO null RNAi lines were inoculated as described with Colletotrichum coccodes hyphal mats, simulating a massive infection titer of anthracnose.

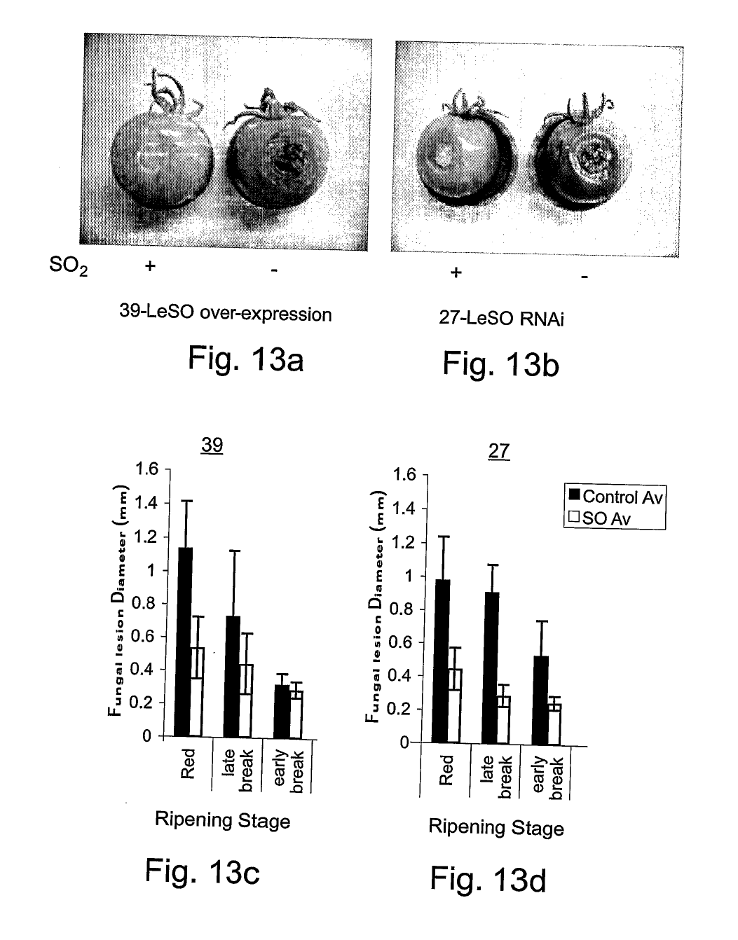

FIGS. 13a and 13b show that the LeSO overexpressing and LeSO null RNAi fruit are equally susceptible to infection by Colletotrichum coccodes, and that both the LeSO overexpressing and LeSO null RNAi fruit were protected from extensive fungal damage by SO₂ fumigation (see also FIGS. 13c and 13d). The fruit was subjected to the following sulfur dioxide schedule: 20 ppm for 4 h followed by a decrease to 2 ppm for 24 h. The fruit were examined 72 h latter. As illustrated by the representative fruits shown in FIGS. 13a and 13b, this regime of fumigation completely arrested fungal growth, irrespective of the genetic background of the tomato. Note the blackish growth of fungus in a wide diameter of increased fungal growth (ie 1 mm beyond the original infection circumference for the indicated time), compared to the whitish fungal mat indicating non-viable fungi in the fumigated (SO₂+) fruits. FIGS. 13c and 13d confirm the equal sensitivity of LeSO over and under-expressing lines to anthracnose, which is more virulent in ripe fruit (“red”).

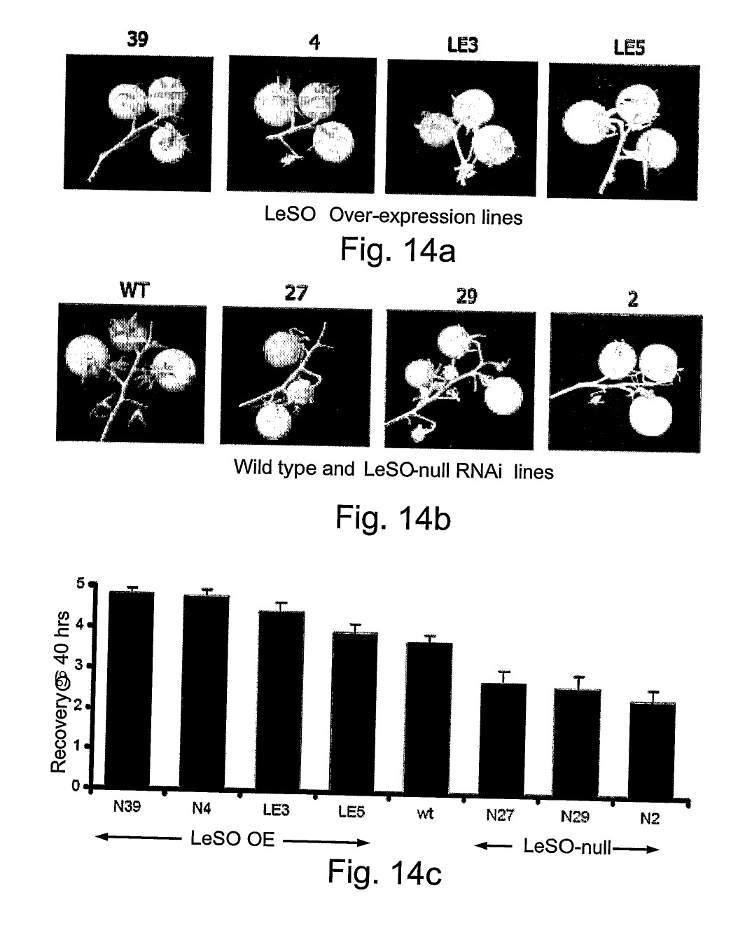

When plants having modified levels of SO, and including fruit and stalk structures are exposed to SO₂ fumigation, the advantage of SO overexpression in plants was observed. Peduncle and calyx of all LeSO overexpression lines (lines 39, 4, Le3 and Le5, FIG. 14a) were significantly less damaged by fumigation (ie showed high recovery rates after 40 hours) than representative wild type or any of the LeSO-null RNAi lines (27, 29 and 2, FIG. 14b), evidenced by the curling of the calyx sepals in all the wild type and SO-null RNAi lines. Quantitative assessment of the damage, representing data from many plants, is shown in FIG. 14c.

These results show the increased resistance of all SO-overexpressing (OE) plants to damage from fumigation with SO₂ at levels sufficient to contain an important fungal disease in plants (anthracnose).

Thus, the results detailed hereinabove clearly show that plant SO activity is crucial for tolerance to sulfite-producing substances, and that up- or down-regulation of SO expression in SO-modified transgenic plants results in plants having enhanced or impaired tolerance to sulfite-related toxicity, respectively.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES

Clough, S. J. and Bent, A. F. (1998) Floral dip: a simplified method for Agrobacterium-mediated transformation of Arabidopsis thaliana. Plant J 16, 735-743.

Eilers, T., Schwarz, G., Brinkmann, H., Witt, C., Richter, T., Nieder, J., Koch, B., Hille, R., Hansch, R. and Mendel, R. R. (2001) Identification and biochemical characterization of Arabidopsis thaliana sulfite oxidase—A new player in plant sulfur metabolism. J. Biol. Chem. 276, 46989-46994.

Hacham, Y., Avrahain, T. and Amir, R. (2002) The N-terminal region of Arabidopsis cystathionine gamma-synthase plays an important regulatory role in methionine metabolism. Plant Physiol. 128, 454-462.

Hansch, R., Lang, C., Riebeseel, E., Lindigkeit, R., Gessler, A., Rennenberg, H. and Mendel, R. R. (2006) Plant sulfite oxidase as novel producer of H2O2—Combination of enzyme catalysis with a subsequent non-enzymatic reaction step. J. Biol. Chem. 281, 6884-6888.

Hansch, R. and Mendel, R. R. (2005) Sulfite oxidation in plant peroxisomes. Photosynth. Res. 86, 337-343.

Lang C. et al (2007) Sulfite oxidase as key enzyme for protecting plants against sulfur dioxide. Plant Cell Environ. 30, 447-55

Hawkesford, M. J. and De Kok, L. J. (2006) Managing sulphur metabolism in plants. Plant Cell Environ. 29, 382-395.

Johnson, et al. Plant Physiology (2001), 127;731-39.

Leustek, T., Martin, M. N., Bick, J. A. and Davies, J. P. (2000) Pathways and regulation of sulfur metabolism revealed through molecular and genetic studies. Annu. Rev. Plant. Phys. 51, 141-165.

Meier et al. Plant Physiol., 1995:107;1105-18

Sessa et al. Plant Mol Biol 1995; 28:145-53

Nowak, K., Luniak, N., Witt, C., Wustefeld, Y., Wachter, A., Mendel, R. R. and Hansch, R. (2004) Peroxisomal localization of sulfite oxidase separates it from chloroplast-based sulfur assimilation. Plant Cell Physiol. 45, 1889-1894.

Sagi, M., Fluhr, R. and Lips, S. H. (1999) Aldehyde oxidase and xanthine dehydrogenase in a flacca tomato mutant with deficient abscisic acid and wilty phenotype. Plant Physiol. 120, 571-577.

Saito, K. (1999) Biosynthesis of cysteine. In Biochemistry and Biotechnology (Singh, B. K., ed.), New York: Marcel Dekker, pp. 267-291.

Saito, K. (2004) Sulfur assimilatory metabolism. The long and smelling road. Plant Physiol. 136, 2443-2450.

Saito, K., Kurosawa, M., Tatsuguchi, K., Takagi, Y. and Murakoshi, I. (1994) Modulation of cysteine biosynthesis in chloroplasts of transgenic tobacco overexpressing cysteine synthase [O-acetylserine(thiol)-lyase]. Plant Physiol. 106, 887-895.

Sanda, S., Leustek, T., Theisen, M. J., Garavito, R. M. and Benning, C. (2001) Recombinant Arabidopsis SQD1 converts udp-glucose and sulfite to the sulfolipid head group precursor UDP-sulfoquinovose in vitro. J. Biol Chem. 276, 3941-3946.

Seo, M., Peeters, A. J. M., Koiwai, H., Oritani, T., Marion-Poll, A., Zeevaart, J. A. D., Koornneef, M., Kamiya, Y. and Koshiba, T. (2000) The Arabidopsis aldehyde oxidase 3 (AA03) gene product catalyzes the final step in abscisic acid biosynthesis in leaves. Proc. Natl. Acad. Sci. USA, 97, 12908-12913.

Wilkinson, J. Q. and Crawford, N. M. (1993) Identification and characterization of a chlorate-resistant mutant of Arabidopsis thaliana with mutations in both nitrate reductase structural genes Nia1 and Nia2. Mol. Gen. Genet. 239, 289-297.

Yesbergenova, Z., Yang, G. H., Oron, E., Soffer, D., Fluhr, R. and Sagi, M. (2005) The plant Mo-hydroxylases aldehyde oxidase and xanthine dehydrogenase have distinct reactive oxygen species signatures and are induced by drought and abscisic acid. Plant J. 42, 862-876.

Yu, B., Xu, C. and Benning, C. (2002) Arabidopsis disrupted in SQD2 encoding sulfolipid synthase is impaired in phosphate-limited growth. Proc. Natl. Acad. Sci. USA 99, 5732-5737.

Zimmermann, P., Hirsch-Hoffmann, M., Hennig, L. and Gruissem, W. (2004) GENEVESTIGATOR. Arabidopsis microarray database and analysis toolbox. Plant Physiol. 136, 2621-2632.

What is claimed is:

1. A method of enhancing tolerance of a plant or plant tissue to a sulfite-producing compound, the method comprising expressing an exogenous sulfite oxidase in the plant or plant tissue, thereby enhancing the tolerance of said plant or plant tissue to the sulfite-producing compound.

2. A method of bioremediation of a sulfite-producing compound, the method comprising contacting the compound with at least one transgenic plant expressing exogenous sulfite oxidase, thereby reducing the concentration of said sulfite-producing compound.

3. The method of claim 2, wherein said at least one transgenic plant is a plurality of plants.

4. The method of claim 2, further comprising the steps of:
(a) assessing a concentration of said sulfite-producing compound prior to said contacting; and/or
(b) assessing a concentration of sulfite-producing compound following said contacting.

5. A device for bioremediation of a sulfite-producing compound, the device comprising an at least partially sealed enclosure comprising at least one transgenic plant expressing an exogenous sulfite oxidase, an inlet for directing the sulfite producing compounds to the transgenic plants within said enclosure, thereby remediating the sulfite-producing compounds, and an outlet for removing remediated sulfite-producing compounds from said enclosure.

6. The device of claim 5, wherein said at least one plant is a plurality of plants.

7. The device of claim 5, wherein said sulfite producing compound is selected from the group consisting of sulfur dioxide, sodium sulfite, sodium bisulfite, sodium metabisulfite, sodium dithionite, sulfur, methionine and cysteine, isothiocyanate and isothioyanate glycosides.

8. The device of claim 5, further comprising a sensor for measuring a level of a sulfite producing compound.

9. A method of promoting tolerance to an ingested sulfite-producing compound in a subject in need thereof, the method comprising orally administering to the subject a therapeutically effective amount of an edible plant material derived from a transgenic plant expressing an exogenous sulfite oxidase, thereby promoting tolerance to said ingested compounds in said subject.

10. A pharmaceutical composition comprising an edible transgenic plant material expressing an exogenous sulfite oxidase and a pharmaceutically acceptable carrier for oral administration, said transgenic plant having elevated levels of a sulfite oxidase catalytic activity as compared to a similar non-transgenic plant.

11. A method of enhancing the post harvest quality of a plant or plant tissue in the presence of sulfur compounds, the method comprising upregulating in the plant an activity or level of a sulfite oxidase so as to increase tolerance to sulfur compounds, thereby enhancing post-harvest quality of the plant or plant tissue in the presence of sulfur compounds.

12. The method of claim 11, wherein said plant tissue is a fruit.

13. A method of monitoring levels of sulfite-producing compounds, the method comprising:
(a) exposing a genetically modified plant having reduced sulfite oxidase catalytic activity as compared to a similar, unmodified plant, to said substance; and
(b) monitoring at least one growth parameter of said genetically modified plant, wherein said at least one growth parameter in said plant or portion thereof is reduced by predetermined levels of said sulfite-producing compounds, thereby monitoring levels of sulfite-producing compounds.

14. An oligonucleotide comprising a nucleic acid sequence capable of specifically hybridizing to a nucleic acid sequence encoding a plant sulfite oxidase and reducing expression of said sulfite oxidase in a plant or plant tissue.

15. The oligonucleotide of claim 14, wherein said nucleic acid sequence encoding the plant sulfite oxidase is as set forth in SEQ ID NO: 16 or 69.

16. The oligonucleotide of claim 14, comprising a nucleotide sequence as set forth in SEQ ID NOs: 75 and 76.

17. The oligonucleotide of claim 14, wherein said oligonucleotide is double stranded.

18. A nucleic acid construct comprising the nucleic acid sequence of claim 14 and a promoter for directing expression of said nucleic acid sequence in a plant.

19. A transgenic plant comprising the nucleic acid construct of claim 18.

20. The method of claims 1, 2, 4, and 6, wherein said plant comprises an exogenous nucleic acid comprising the sequence as set forth in SEQ ID NO: 16 and 69-73.

21. The method of claims 1, 2, 9 and 11, wherein said plant comprises an exogenous nucleic acid encoding a polypeptide having a sulfite oxidase catalytic activity having an amino acid sequence as set forth by SEQ ID NO: 1 and 76-79.

22. The method of claims 1, 2, 9 and 13, wherein said sulfite producing compound is a gas.

23. The method of claim 22, wherein said sulfite producing compound is a liquid.

24. The method of claims 1, 2, 9 and 13, wherein said sulfite producing compound is selected from the group consisting of a sulfur dioxide, sulfur, sodium sulfite, sodium bisulfite, sodium metabisulfite, sodium dithionite, methionine, cysteine, isothiocyanate and isothioyanate glycosides.

25. The method of claims 1, 2, 9 and 13 wherein said sulfite producing compound is a sulfur dioxide.

26. The method of claim 25, wherein a concentration of said sulfur dioxide is less than or equal to 1 ppm.

27. The method of claim 25, wherein a concentration of said sulfur dioxide is between 1 to 2 ppm.

28. The method of claim 25, wherein a concentration of said sulfur dioxide is greater than 2 ppm.

29. The method of claims 1, 2, 9 and 13 wherein said expressing is effected in a tissue specific manner.

30. The method of claim 29, wherein said tissue is selected from the group consisting of a leaf, a fruit, a root, a stem and a flower of said plant.

31. The method of claims 1, 2, 5, 6, 11 and 13 wherein the plant is selected from the group consisting of plantation plants, orchard plants, field crop plants and ornamental plants.
